SEQUENCE LISTING (40 sequences)

SEQ ID NO: 1 - protein, 510 aa, Arabidopsis thaliana
MILVLASLFAVLILNVLLWRWLKASACKAQRLPPGPPRLPILGNLLQLGPLPHRDLASLCDKYGPLVYLRLGNVDAITTNDPDTIREILLRQDDVFSSRPKTLAAVHLAYGCGDVALAPMGPHWKRMRRICMEHLLTTKRLESFTTQRAEEARYLIRDVFKRSETGKPINLKEVLGAFSMNNVTRMLLGKQFFGPGSLVSPKEAQEFLHITHKLFWLLGVIYLGDYLPFWRWVDPSGCEKEMRDVEKRVDEFHTKIIDEHRRAKLEDEDKNGDMDFVDVLLSLPGENGKAHMEDVEIKALIQDMIAAATDTSAVTNEWAMAEAIKQPRVMRKIQEELDNVVGSNRMVDESDLVHLNYLRCVVRETFRMHPAGPFLIPHESVRATTINGYYIPAKTRVFINTHGLGRNTKIWDDVEDFRPERHWPVEGSGRVEISHGPDFKILPFSAGKRKCPGAPLGVTMVLMALARLFHCFEWSSPGNIDTVEVYGMTMPKAKPLRAIAKPRLAAHLYT

SEQ ID NO: 2 - protein, 711 aa, Arabidopsis thaliana
MAFSHDNRVRFKDEGKPLSSEYGYGRKARPSLDRVFKNVKWGFKKPLSFPSHKDPDHKETSSVTRKNIINPQDSFLQNWNKIFLFACVVALAIDPLFFYIPIVDSARHCLTLDSKLEIAASLLRTLIDAFYIIHIVFQFRTAYIAPSSRVFGRGELVDDAKAIALKYLSSYFIIDLLSILPLPQIVVLAVIPSVNQPVSLLTKDYLKFSIIAQYVPRILRMYPLYTEVTRTSGIVTETAWAGAAWNLSLYMLASHVFGALWYLISVEREDRCWQEACEKTKGCNMKFLYCENDRNVSNNFLTTSCPFLDPGDITNSTIFNFGIFTDALKSGVVESHDFWKKFFYCFWWGLRNLSALGQNLQTSKFVGEIIFAISICISGLVLFALLIGNMQKYLESTTVREEEMRVRKRDAEQWMSHRMLPEDLRKRIRRYEQYRWQETRGVEEETLLRNLPKDLRRDIKRHLCLDLLKKVPLFEIMDEQLLDAVCDRLRPVLYTENSYVIREGDPVGEMLFVMRGRLVSATTNGGRSGFFNAVNLKASDFCGEDLLPWALDPQSSSHFPISTRTVQALTEVEAFALTAEDLKSVASQFRRLHSKQLQHTFRFYSVQWRTWSVSFIQAAWRRYCRRKLAKSLRDEEDRLREALASQDKEHNAATVSSSLSLGGALYASRFASNALHNLRHNISNLPPRYTLPLLPQKPTEPDFTANHTTDP

SEQ ID NO: 3 - protein, 490 aa, Arabidopsis thaliana
MAETTSWIPVWFPLMVLGCFGLNWLVRKVNVWLYESSLGENRHYLPPGDLGWPFIGNMLSFLRAFKTSDPDSFTRTLIKRYGPKGIYKAHMFGNPSIIVTTSDTCRRVLTDDDAFKPGWPTSTMELIGRKSFVGISFEEHKRLRRLTAAPVNGHEALSTYIPYIEENVITVLDKWTKMGEFEFLTHLRKLTFRIIMYIFLSSESENVMDALEREYTALNYGVRAMAVNIPGFAYHRALKARKTLVAAFQSIVTERRNQRKQNILSNKKDMLDNLLNVKDEDGKTLDDEEIIDVLLMYLNAGHESSGHTIMWATVFLQEHPEVLQRAKAEQEMILKSRPEGQKGLSLKETRKMEFLSQVVDETLRVITFSLTAFREAKTDVEMNGYLIPKGWKVLTWFRDVHIDPEVFPDPRKFDPARWDNGFVPKAGAFLPFGAGSHLCPGNDLAKLEISIFLHHFLLKYQVKRSNPECPVMYLPHTRPTDNCLARISYQ

SEQ ID NO: 4 - protein, 442 aa, Arabidopsis thaliana
MADICYEDETSACESRPLWSSRKWRIGVQRFRMSPSEMNPTASTTEEEDKSEGIYNKRNKQEEYDFMNCASSSPSQSSPEEESVSLEDSDVSISDGNSSVNDVAVIPSKKTVKETDLRPRYGVASVCGRRRDMEDAVALHPSFVRKQTEFSRTRWHYFGVYDGHGCSHVAARCKERLHELVQEEALSDKKEEWKKMMERSFTRMDKEVVRWGETVMSANCRCELQTPDCDAVGSTAVVSVITPEKIIVANCGDSRAVLCRNGKAVPLSTDHKPDRPDELDRIQEAGGRVIYWDGARVLGVLAMSRAIGDNYLKPYVTSEPEVTVTDRTEEDEFLILATDGLWDVVTNEAACTMVRMCLNRKSGRGRRRGETQTPGRRSEEEGKEEEEKVVGSRKNGKRGEITDKACTEASVLLTKLALAKHSSDNVSVVVIDLRRRRKRHVA

SEQ ID NO: 5 - protein, 428 aa, Arabidopsis thaliana
MSVSKASRTQHSLVPLATLIGRELRSEKVEKPFVKYGQAALAKKGEDYFLIKTDCERVPGDPSSAFSVFGIFDGHNGNSAAIYTKEHLLENVVSAIPQGASRDEWLQALPRALVAGFVKTDIEFQQKGETSGTTVTFVIIDGWTITVASVGDSRCILDTQGGVVSLLTVDHRLEENVEERERITASGGEVGRLNVFGGNEVGPLRCWPGGLCLSRSIGDTDVGEFIVPIPHVKQVKLPDAGGRLIIASDGIWDILSSDVAAKACRGLSADLAAKLVVKEALRTKGLKDDTTCVVVDIVPSGHLSLAPAPMKKQNPFTSFLSRKNHMDTNNKNGNKLSAVGVVEELFEEGSAVLADRLGKDLLSNTETGLLKCAVCQIDESPSEDLSSNGGSIISSASKRWEGPFLCTICKKKKDAMEGKRPSKGSVTT

SEQ ID NO: 6 - protein, 510 aa, Arabidopsis thaliana
MDLTDVIIFLFALYFINLWWRRYFSAGSSQCSLNIPPGPKGWPLVGNLLQVIFQRRHFVFLMRDLRKKYGPIFTMQMGQRTMIIITDEKLIHEALVQRGPTFASRPPDSPIRLMFSVGKCAINSAEYGSLWRTLRRNFVTELVTAPRVKQCSWIRSWAMQNHMKRIKTENVEKGFVEVMSQCRLTICSILICLCFGAKISEEKIKNIENVLKDVMLITSPTLPDFLPVFTPLFRRQVREARELRKTQLECLVPLIRNRRKFVDAKENPNEEMVSPIGAAYVDSLFRLNLIERGGELGDEEIVTLCSEIVSAGTDTSATTLEWALFHLVTDQNIQEKLYEEVVGVVGKNGVVEEDDVAKMPYLEAIVKETLRRHPPGHFLLSHAAVKDTELGGYDIPAGAYVEIYTAWVTENPDIWSDPGKFRPERFLTGGDGVDADWTGTRGVTMLPFGAGRRICPAWSLGILHINLMLARMIHSFKWIPVPDSPPDPTETYAFTVVMKNSLKAQIRSRT

SEQ ID NO: 7 - protein, 433 aa, Arabidopsis thaliana
MAILFCFFLVSLVTLVSSIFFKQIKNTKFNLPPSPPSLPIIGNLHHLTGLPHRCYHKLSIKYGPVILLHLGFVPVVVISLSEAAEAVLKTHDLECCSRPKTVGTGKLSYGFKDISFVPYSEYWREMRKLAVTELFSLKKEGIEELVTAATTAIGSFTFSDFFPSGLGRFLDCLFRTQTNINKVSEKLDAFYQHVIDDHLKPSTLDSSGDIVALMLDMIKKKGHKDDFKLNVDNIKAVLMNIFLAGIDTGAITMIWAMTELVKKPLVMKRAQENIRGVLGLKRDRITEEDLCKFDCLKHIVKETLRLHPPVPFLVPRETISHIKIQGYDIPPKTQIQVNRWTDPEEFRPERFANTCVDFRGQHFDFLPFGSGRRICPAISMAIATVELGLMNLLDFFDWRLPDGMKVEDIDIEEAGNVTVVKKLLIYLVPLQRH

SEQ ID NO: 8 - protein, 491 aa, Arabidopsis thaliana
MGLCHSKIDKTTRKETGATSTATTTVERQSSGRLRRPRDLYSGGEISEIQQVVGRLVGNGSSEIACLYTQQGKKGTNQDAMLVWENFCSRSDTVLCGVFDGHGPFGHMVSKRVRDMLPFTLSTQLKTTSGTEQSSSKNGLNSAPTCVDEEQWCELQLCEKDEKLFPEMYLPLKRALLKTCQQMDKELKMHPTINCFCSGTTSVTVIKQGKDLVVGNIGDSRAVLATRDQDNALVAVQLTIDLKPDLPSESARIHRCKGRVFALQDEPEVARVWLPNSDSPGLAMARAFGDFCLKDYGLISVPDINYHRLTERDQYIILATDGVWDVLSNKEAVDIVASAPSRDTAARAVVDTAVRAWRLKYPTSKNDDCAVVCLFLEDTSAGGTVEVSETVNHSHEESTESVTITSSKDADKKEEASTETNETVPVWEIKEEKTPESCRIESKKTTLAECISVKDDEEWSALEGLTRVNSLLSIPRFFSGELRSSSWRKWL

SEQ ID NO: 9 - protein, 537 aa, Arabidopsis thaliana
MSFTTSLPYPFHILLVFILSMASITLLGRILSRPTKTKDRSCQLPPGPPGWPILGNLPELFMTRPRSKYFRLAMKELKTDIACFNFAGIRAITINSDEIAREAFRERDADLADRPQLFIMETIGDNYKSMGISPYGEQFMKMKRVITTEIMSVKTLKMLEAARTIEADNLIAYVHSMYQRSETVDVRELSRVYGYAVTMRMLFGRRHVTKENVFSDDGRLGNAEKHHLEVIFNTLNCLPSFSPADYVERWLRGWNVDGQEKRVTENCNIVRSYNNPIIDERVQLWREEGGKAAVEDWLDTFITLKDQNGKYLVTPDEIKAQCVEFCIAAIDNPANNMEWTLGEMLKNPEILRKALKELDEVVGRDRLVQESDIPNLNYLKACCRETFRIHPSAHYVPSHLARQDTTLGGYFIPKGSHIHVCRPGLGRNPKIWKDPLVYKPERHLQGDGITKEVTLVETEMRFVSFSTGRRGCIGVKVGTIMMVMLLARFLQGFNWKLHQDFGPLSLEEDDASLLMAKPLHLSVEPRLAPNLYPKFRP

SEQ ID NO: 10 - protein, 476 aa, Arabidopsis thaliana
MLEIITVRKVFLIGFLILILNWVWRAVNWVWLRPKRLEKYLKKQGFSGNSYRILMGDMRESNQMDQVAHSLPLPLDADFLPRMMPFLHHTVLKHGKKCFTWYGPYPNVIVMDPETLREIMSKHELFPKPKIGSHNHVFLSGLLNHEGPKWSKHRSILNPAFRIDNLKSILPAFNSSCKEMLEEWERLASAKGTMELDSWTHCHDLTRNMLARASFGDSYKDGIKIFEIQQEQIDLGLLAIRAVYIPGSKFLPTKFNRRLRETERDMRAMFKAMIETKEEEIKRGRAGQNVTSSLFVWTLVALSQHQDWQNKARDEISQAFGNNEPDFEGLSHLKVVTMILHEVLRLYSPAYFTCRITKQEVKLERFSLPEGVVVTIPMLLVHHDSDLWGDDVKEFKPERFANGVAGATKGRLSFLPFSSGPRTCIGQNFSMLQAKLFLAMVLQRFSVELSPSYTHAPFPAATTFPQHGAHLIIRKL

SEQ ID NO: 11 - protein, 728 aa, Arabidopsis thaliana
MSSNATGMKKRSCFGLFNVTSRGGGKTKNTSKSFREGVKIGSEGLKTIGKSFTSGVTRAVFPEDLRVSEKKIFDPQDKTLLLWNRMFVISCILAVSVDPLFFYLPIVDNSKNCIGIDSKLAVTTTTLRTIIDVFYLTRMALQFRTAYIAPSSRVFGRGELVIDPAKIAERYLTRYFIVDFLAVLPLPQIAVWKFLHGSKGTDVLPTKQALLHIVITQYIPRFVRFIPLTSELKKTAGAFAEGAWAGAAYYLLWYMLASHITGAFWYMLSVERNDTCLRSACKVQPDPKVCVQILYCGSKLMSSRETDWIKSVPDLFKNNCSAKSDESKFNYGIYSQAVSSGIVSSTTFFSKFCYCLWWGLQNLSTLGQGLQTSTYPGEVLFSIAIAVAGLLLFALLIGNMQTYLQSLTVRLEEMRIKRRDSEQWMHHRSLPQNLRERVRRYDQYKWLETRGVDEENIVQSLPKDLRRDIKRHLCLNLVRRVPLFANMDERLLDAICERLKPSLYTESTYIVREGDPVNEMLFIIRGRLESVTTDGGRSGFFNRGLLKEGDFCGEELLTWALDPKAGSNLPSSTRTVKALTEVEAFALEAEELKFVASQFRRLHSRQVQQTFRFYSQQWRTWAACFIQAAWRRHLRRKIAELRRKEEEEEEMDYEDDEYYDDNMGGMVTRSDSSVGSSSTLRSTVFASRFAANALKGHKLRVTESSKSLMNLTKPSEPDFEALDTDDLN

SEQ ID NO: 12 - protein, 522 aa, Arabidopsis thaliana
MNVLISAVVWVYTHLRLSDVALALVGLFLLSYLREKLVSKGGPVMWPVLGIIPMLALNKHDLFTWCTRCVVRSGGTFHYRGIWFGGAYGIMTADPANVEHILKTNFKNYPKGAFYRERFRDLLEDGIFNADDELWKEERRVAKTEMHSSRFLEHTFTTMRDLVDQKLVPLMENLSTSKRVFDLQDLLLRFTFDNICISAFGVYPGSLETGLPEIPFAKAFEDATEYTLARFLIPPFVWKPMRFLGIGYERKLNNAVRIVHAFANKTVRERRNKMRKLGNLNDYADLLSRLMQREYEKEEDTTRGNYFSDKYFREFCTSFIIAGRDTTSVALVWFFWLVQKHPEVEKRILREIREIKRKLTTQETEDQFEAEDFREMVYLQAALTESLRLYPSVPMEMKQALEDDVLPDGTRVKKGARIHYSVYSMGRIESIWGKDWEEFKPERWIKEGRIVSEDQFKYVVFNGGPRLCVGKKFAYTQMKMVAAAILMRYSVKVVQGQEIVPKLTTTLYMKNGMNVMLQPRDW

SEQ ID NO: 13 - protein, 186 aa, Arabidopsis thaliana
MFNKNQGSNGGSSSNVGIGADSPYLQKARSGKTEIRELEAVFKKFDVNGDGKISSKELGAIMTSLGHEVPEEELEKAITEIDRKGDGYINFEEFVELNTKGMDQNDVLENLKDAFSVYDIDGNGSISAEELHEVLRSLGDECSIAECRKMIGGVDKDGDGTIDFEEFKIMMTMGSRRDNVMGGGPR

SEQ ID NO: 14 - protein, 166 aa, Arabidopsis thaliana
MSHKVSKKLDEEQINELREIFRSFDRNKDGSLTQLELGSLLRALGVKPSPDQFETLIDKADTKSNGLVEFPEFVALVSPELLSPAKRTTPYTEEQLLRLFRIFDTDGNGFITAAELAHSMAKLGHALTVAELTGMIKEADSDGDGRINFQEFAKAINSAAFDDIWG

SEQ ID NO: 15 - protein, 372 aa, Arabidopsis thaliana
KVEEMTFTQLFSPQRIEATKALRMKKVQELVNFLSESSERGEAVDISRASFVTALNIISNILFSVNLGSYDSKNSSAFQEMVIGYMESIGNPDVSNFFPFMRLLDLQGNSKKMKEYSGKLLQVFREFYDARILENSSRIDEKDVSSRDFLDALIDLQQGDESEINIDEIEHLLLDMFLAGTDTNSSTVEWAMTELLGNPKTMTKVQDEINRVIRQNGDVQESHISKLPYLQAVIKETFRLHPAAPFLLPRKAERDVDILGFHVPKDSHVLVNVWAIGRDPNVWENPTQFEPERFLGKDIDVKGTNYELTPFGAGRRICPGLPLALKTVHLMLASLLYTFEWKLPNGVGSEDLDMGETFGLTVHKTNPLLACL

SEQ ID NO: 16 - protein, 1096 aa, Arabidopsis thaliana
MSVDRRNWLKHGCNLRLVILVLWLVCYVSNGQTIGDTSDFNNPAVLPLVTQMVYRSLSNSTAALNRELGIRAKFCVKDPDADWNRAFNFSSNLNFLSSCIKKTQGSIGKRICTAAEMKFYFNGFFNKTNNPGYLKPNVNCNLTSWVSGCEPGWGCSVDPTEQVDLQNSKDFPERRRNCMPCCEGFFCPRGLTCMIRKEFKTLTLACILINYHQDDQTTLVEVQMYGPISDPAVKYSVPQDHIVQPQPKKYLVIVGTTAGWGLLLRNLTSCNPNTANQNMHAFGIMVIAAVSTILLIIYNCSDQILTTRERRQAKSREAAVKKARAHHRWKAAREAAKKHVSGIRAQITRTFSGKRANQDGDTNKMLGRGDSSEIDEAIDMSTCSSPASSSAAQSSYENEDHAAAGSNGRASLGIEGKRVKGQTLAKIKKTQSQIFKYAYDRIEKEKAMEQENKNLTFSGIVKMATNSETRKRHLMELSFKDLTLTLKSNGKQVLRCVTGSMKPGRITAVMGPSGAGKTSLLSALAGKAVGCKLSGLILINGKQESIHSYKKIIGFVPQDDVVHGNLTVEENLWFHAKCRLPADLSKADKVLVVERIIDSLGLQAVRSSLVGTVEKRGISGGQRKRVNVGLEMVMEPSVLFLDEPTSGLDSASSQLLLRALRHEALEGVNICMVVHQPSYTLFKTFNDLVLLAKGGLTVYHGSVNKVEEYFSGLGIHVPDRINPPDYYIDVLEGVVISMGNSGIGYKELPQRWMLHKGYSVPLDMRNNSAAGLETNPDLGTNSPDNAEQTFARELWRDVKSNFRLRRDKIRHNFLKSRDLSHRRTPSTWLQYKYFLGRIAKQRMREAQLQATDYLILLLAGACLGSLIKASDESFGAPALLCKIAALRSFSLDKLHYWRESASGMSSSACFLAKDTIDIFNILVKPLVYLSMFYFFTNPRSTFFDNYIVLVCLVYCVTGIAYALAIFLQPSTAQLFSVLLPVVLTLVATQPKNSELIRIIADLSYPKWALEAFVIGNAQKYYGVWMITRCGSLMKSGYDINKWSLCIMILLLVGLTTRGVAFVGMLILQKKYLKLPVEEELHEFCGWLDLTMIIALL

SEQ ID NO: 17 - protein, 148 aa, Arabidopsis thaliana
MSKDGLSNDQVSSMKEAFMLFDTDGDGKIAPSELGILMRSLGGNPTESQLKSIITTENLSSPFDFNRFLDLMAKHLKTEPFDRQLRDAFKVLDKEGTGFVAVADLRHILTSIGEKLQPSEFDEWIKEVDVGSDGKIRYEDFIARMVAK

SEQ ID NO: 18 - protein, 715 aa, Arabidopsis thaliana
MTEVASSVVYEVLGRRAQDVDEPIMDYIINVLADEDFDFGEEGEGAFDAVGELLVAAECVSDFEECRLVCSKLSDKFGKHGLVKPTPTVRSLAMPVRMNDGMDDGPVKKKKPEPVDGPLLTERDLAKIERRKKKDDRQRELQYQQHVAEMEAAKAGMPTVSVNHDTGGGSAIRDIHMDNFNVSVGGRDLIVDGSITLSFGRHYGLVGRNGTGKTTFLRYMAMHAIEGIPTNCQILHVEQEVVGDKTTALQCVLNTDIERTKLLEEEIQILAKQRETEEPTAKDGMPTKDTVEGDLMSQRLEEIYKRLDAIDAYTAEARAASILAGLSFTPEMQLKATNTFSGGWRMRIALARALFIEPDLLLLDEPTNHLDLHAVLWLETYLTKWPKTFIVVSHAREFLNTVVTDIIHLQNQKLSTYKGNYDIFERTREEQVKNQQKAFESSERSRSHMQAFIDKFRYNAKRASLVQSRIKALDRLAHVDQVINDPDYKFEFPTPDDKPGPPIISFSDASFGYPGGPLLFRNLNFGIDLDSRIAMVGPNGIGKSTILKLISGDLQPSSGTVFRSAKVRVAVFSQHHVDGLDLSSNPLLYMMRCYPGVPEQKLRSHLGSLGVTGNLALQPMYTLSGGQKSRVAFAKITFKKPHLLLLDEPSNHLDLDAVEALIQGLVLFQGGICMVSHDEHLISGSVDELWVVSDGRIAPFHGTFHDYKKLLQSST

SEQ ID NO: 19 - protein, 157 aa, Arabidopsis thaliana
MSKNVSRNCLGSMEDIKKVFQRFDKNNDGKISIDELKDVIGALSPNASQEETKAMMKEFDLDGNGFIDLDEFVALFQISDQSSNNSAIRDLKEAFDLYDLDRNGRISANELHSVMKNLGEKCSIQDCQRMINKVDSDGDGCVDFEEFKKMMMINGSA

SEQ ID NO: 20 - protein, 149 aa, Arabidopsis thaliana
MADQLTDEQISEFKEAFSLFDKDGDGCITTKELGTVMRSLGQNPTEAELQDMINEVDADGNGTIDFPEFLNLMAKKMKDTDSEEELKEAFRVFDKDQNGFISAAELRHVMTNLGEKLTDEEVEEMIREADVDGDGQINYEEFVKIMMAK

SEQ ID NO: 21 - protein, 1434 aa, Arabidopsis thaliana
MAAMLGRDEDPVGALSGRVSLASTSHRSLVGASKSFRDVFMPQTDEVFGRSERREEDDMELRWAAIERLPTFDRLRKGMLPQTSANGKIELEDIDLTRLEPKDKKHLMEMILSFVEEDNEKFLRDLRERTDRVGIEVPKIEVRYENISVEGDVRSASRALPTLFNVTLNTLESILGFFHLLPSKRKKIQILKDISGIVKPSRMTLLLGPPSSGKTTLLQALAGKLDDTLQMSGRITYCGHEFREFVPQKTCAYISQHDLHFGEMTVREILDFSGRCLGVGSRYQLMSELSRREKEEGIKPDPKIDAFMKSIAISGQETSLVTDYVLKILGLDICADILAGDVMRRGISGGQKKRLTTGEMLVGPARALFMDEISTGLDSSTTFQICKFMRQLVHISDVTMIISLLQPAPETFELFDDIILLSEGQIVYQGPRDNVLEFFEYFGFQCPERKGVADFLQEVTSKKDQEQYWNKREQPYNYVSVSDFSSGFSTFHTGQKLTSEFRVPYDKAKTHSAALVTQKYGISNWELFKACFDREWLLMKRNSFVYVFKTVQITIMSLITMTVYLRTEMHVGTVRDGQKFYGAMFFSLINVMFNGLAELAFTVMRLPVFYKQRDFLFYPPWAFALPAWLLKIPLSLIESGIWIGLTYYTIGFAPSAARFLGAIGRTEVISNSIGTFTLLIVFTLGGFIIAKDDIRPWMTWAYYMSPMMYGQTAIVMNEFLDERWSSPNYDTRINAKTVGEVLLKSRGFFTEPYWFWICIVALLGFSLLFNLFYILALMYLNPLGNSKATVVEEGKDKQKGENRGTEGSVVELNSSSNKGPKRGMVLPFQPLSLAFNNVNYYVDMPSEMKAQGVEGDRLQLLRDVGGAFRPGILTALVGVSGAGKTTLMDVLAGRKTGGYIEGSISISGYPKNQTTFARVSGYCEQNDIHSPHVTVYESLIYSAWLRLSTDIDIKTRELFVEEVMELVELKPLRNSIVGLPGVDGLSTEQRKRLTIAVELVANPSIIFMDEPTSGLDARAAAIVMRTVRNTVDTGRTVVCTIHQPSIDIFESFDELLLMKRGGQVIYAGSLGHHSQKLVEYFEAVEGVPKINDGYNPATWMLDVTTPSMESQMSLDFAQIFSNSSLYRRNQELIKDLSTPPPGSKDVYFKTKYAQSFSTQTKACFWKQYWSYWRHPQYNAIRFLMTVVIGVLFGLIFWQIGTKTENEQDLNNFFGAMYAAVLFLGALNAATVQPAIAIERTVFYREKAAGMYSAIPYAISQVAVEIMYNTIQTGVYTLILYSMIGCNWTMAKFLWFYYYMLTSFIYFTLYGMMLMALTPNYQIAGICMSFFLSLWNLFSGFLIPRPQIPIWWRWYYWATPVAWTLYGLITSQVGDKDSMVHISGIGDIDLKTLLKEGFGFEHDFLPVVAVVHIAWILLFLFVFAYGIKFLNFQRR

SEQ ID NO: 22 - protein, 263 aa, Arabidopsis thaliana
MPSLWSNESDGSLREHLVDVVVSGSEPKIRVHDLTRVADDGSRILKGVTIDIPKGMIVGVIGPSGSGKSTFLRSLNRLWEPPESTVFLDGEDITNVDVIALRRRVGMLFQLPVLFQGTVADNVRYGPNLRGEKLSDEEVYKLLSLADLDASFAKKTGAELSVGQAQRVALARTLANEPEVLLLDEPTSALDPISTENIEDVIVKLKKQRGITTVIVSHSIKQIQKVADIVCLVVDGEIVEVLKPSELSHATHPMAQRFLQLSS

SEQ ID NO: 23 - protein, 659 aa, Arabidopsis thaliana
MAQQVLGCTSRPIRVSLHRCSVITTSDTIRRKNLRFVRNPRLSFSLQSSTRNYRLPSINCSTVNGAVAETAEYYEGEGDNVSLAEKIRQCIDFLRTILPGGSWWSFSDEVDGRFIAKPVTVWRALSRMWELVAEDRWVIFAAFSTLIVAAVRGSSLLSEITIPHFLTASIFSAQSGDIAVFHRNVKLLDISFFDSQTVGDLTSRLGSDCQQVSRVIGNDLNMIFRNVLQGTGALIYLLILSWPLGLCTLVICCILAAVMFVYGMYQKKTAKLIQEITASANEVAQETYSLMRTVRVYGTEKQEFKRYNHWLQRLADISLRQSAAYGIWNWSFNTLYHATQIIAVLVGGLSILAGQITAEQLTKFLLYSEWLIYATWWVGDNLSSLMQSVGASEKVFQMMDLKPRTRLQRLTGHIEFVDVSFSYPSRDEVAVVQNVNISVHPGEVVAIVGLSGSGKSTLVNLLLQLYEPTSGQILLDGVPLKELDVKWLRQRIGYVGQEPKLFRTDISSNIKYGCDRNISQEDIISAAKQAYAHDFITALPNGYNTIVDDDLLSGGQKQRIAIARAILRDPRILILDEATSALDAESEHNVKGVLRSIGNDSATKRSVIVIAHRLSTIQAADRIVAMDSGRVVEMGSHKELLSKDGLYARLTKRQNDAVL

SEQ ID NO: 24 - protein, 324 aa, Arabidopsis thaliana
MDRERYDKVIEACSLSKDLEILSFGDQTVIGERGINLSGGQKQRIHIARALYQDADIYLFDDPFSAVDAHTGSHLFKEALRGLLCSKSVIYVTHQVEFLPSADLTLVMKDGRISQAGKYNDILISGTDFRELIGAHQESLAVVGSADASSVSENSALDEENGVVRDDIGFDGKQESQDLKNDKLDSGEPQRQFVQEEERAKGSVALDVYWKYITLAYGGALVPFILLGQILFQLLQIGSNYWMAWATPISEDVQAPVKLSTLMVVYVALAFGSSLCILVRATLLVTAGYKTATELFHKMHHCIFRSPMSFKIAKTCSKTCIYSS

SEQ ID NO: 25 - protein, 609 aa, Arabidopsis thaliana
MSNDSCNIKKLLGLKQKPSDETRSTEERTILSGVTGMISPGEFMAVLGPSGSGKSTLLNAVAGRLHGSNLTGKILINDGKITKQTLKRTGFVAQDDLLYPHLTVRETLVFVALLRLPRSLTRDVKLRAAESVISELGLTKCENTVVGNTFIRGISGGERKRVSIAHELLINPSLLVLDEPTSGLDATAALRLVQTLAGLAHGKGKTVVTSIHQPSSRVFQMFDTVLLLSEGKCLFVGKGRDAMAYFESVGFSPAFPMNPADFLLDLANGVCQTDGVTEREKPNVRQTLVTAYDTLLAPQVKTCIEVSHFPQDNARFVKTRVNGGGITTCIATWFSQLCILLHRLLKERRHESFDLLRIFQVVAASILCGLMWWHSDYRDVHDRLGLLFFISIFWGVLPSFNAVFTFPQERAIFTRERASGMYTLSSYFMAHVLGSLSMELVLPASFLTFTYWMVYLRPGIVPFLLTLSVLLLYVLASQGLGLALGAAIMDAKKASTIVTVTMLAFVLTGGYYVNKVPSGMVWMKYVSTTFYCYRLLVAIQYGSGEEILRMLGCDSKGKQGASAATSAGCRFVEEEVIGDVGMWTSVGVLFLMFFGYRVLAYLALRRIKH

SEQ ID NO: 26 - protein, 511 aa, Arabidopsis thaliana
MEEMTPAVAMTLSLAANTMCESSPVEITQLKNVTDAADLLSDSENQSFCNGGTECTMEDVSELEEVGEQDLLKTLSDTRSGSSNVFDEDDVLSVVEDNSAVISEGLLVVDAGSELSLSNTAMEIDNGRVLATAIIVGESSIEQVPTAEVLIAGVNQDTNTSEVVIRLPDENSNHLVKGRSVYELDCIPLWGTVSIQGNRSEMEDAFAVSPHFLKLPIKMLMGDHEGMSPSLTHLTGHFFGVYDGHGGHKVADYCRDRLHFALAEEIERIKDELCKRNTGEGRQVQWDKVFTSCFLTVDGEIEGKIGRAVVGSSDKVLEAVASETVGSTAVVALVCSSHIVVSNCGDSRAVLFRGKEAMPLSVDHKPDREDEYARIENAGGKVIQWQGARVFGVLAMSRSIGDRYLKPYVIPEPEVTFMPRSREDECLILASDGLWDVMNNQEVCEIARRRILMWHKKNGAPPLAERGKGIDPACQAAADYLSMLALQKGSKDNISIIVIDLKAQRKFKTRT

SEQ ID NO: 27 - protein, 163 aa, Arabidopsis thaliana
MANTNLESTNKSTTPSTDMELKKVFDKFDANGDGKISVSELGNVFKSMGTSYTEEELNRVLDEIDIDCDGFINQEEFATICRSSSSAVEIREAFDLYDQNKNGLISSSEIHKVLNRLGMTCSVEDCVRMIGHVDTDGDGNVNFEEFQKMMSSPELVKGTVANS

SEQ ID NO: 28 - protein, 211 aa, Arabidopsis thaliana
MHLDHFFLKLWDPLGTLSKIYSILVYPLNIVVTTYLKWFIEPLPQNYMPYQTPHQQVSSSFGMYPQQPRPPVFVRRQNEPPNIRMVNNNALQHGGMQTSVNQSPLRFRLPNINNRQAPVRPNANAIIPPQGEILGCRRRSYPSRFEFGQSSSSSAQRRRIVPASENVGSTSINHGNAEQRLFFLHFFLLTYHTFFKYLFHIYTLPFFYLLI

SEQ ID NO: 29 - protein, 238 aa, Arabidopsis thaliana
MEDYHVAKFTNFNGNELGLFAIFDGHKGDHVAAYLQKHLFSNILKDGEFLVDPRRAIAKAYENTDQKILADNRTDLESGGSTAVTAILINGKALWIANVGDSRAIVSSRGKAKQMSVDHDPDDDTERSMIESKGGFVTNRPGDVPRVNGLLAVSRVFGDKNLKAYLNSEPEIKDVTIDSHTDFLILASDGISKVMSNQEAVDVAKKLKDPKEAARQVVAEALKRNSKDDISCIVVRFR

SEQ ID NO: 30 - protein, 140 aa, Arabidopsis thaliana
MRKSYLHNYRSSDYLTISMLMADNSSGQSLSHRKPPTSSPSSISTTVSSPKSPFRLRFEKPPSRFGKRPTRLDIPIGVAGFVAPISSSADVAMTSREECREVEREGDGYFVYCKRGRREAREVISSWTPMIMLDEQTKKC

SEQ ID NO: 31 - protein, 514 aa, Arabidopsis thaliana
MGCAYSKTCIGQICATKENSIRQTHQQAPSRGGTRATAAAAAVEEDNPVFNFSSDAVDDVDNDEIHQLGLSRDQEWGITRLSRVSSQFLPPDGSRVVKVPSCNYELRCSFLSQRGYYPDALDKANQDSFAIHTPFGSNSDDHFFGVFDGHGEFGAQCSQFVKRRLCENLLRHGRFRVDPAEACNSAFLTTNSQLHADLVDDSMSGTTAITVMVRGRTIYVANAGDSRAVLAEKRDGDLVAVDLSIDQTPFRPDELERVKLCGARVLTLDQIEGLKNPDVQCWGTEEDDDGDPPRLWVPNGMYPGTAFTRSIGDSIAETIGVVANPEIAVVELTPDNPFFVVASDGVFEFISSQTVVDMVAKHKDPRDACAAIVAESYRLWLQYETRTDDITIIVVHIDGLKDDAPRQLSSTGTQLQPPIPQVVELTGSESPSTFGWNSKNQRVRHDLSRARIRAIENSLENGHAWVPPSPAHRKTWEEEVRVLVCFVFAQPIRNASSHSYIRRLNAGFSRAGTH

SEQ ID NO: 32 - protein, 290 aa, Arabidopsis thaliana
MAGREILHKMKVGLCGSDTGRGKTKVWKNIAHGYDFVKGKAGHPMEDYVVSEFKKVDGHDLGLFAIFDGHLGHDVAKYLQTNLFDNILKEKDFWTDTKNAIRNAYISTDAVILEQSLKLGKGGSTAVTGILIDGKTLVIANVGDSRAVMSKNGVASQLSVDHEPSKEQKEIESRGGFVSNIPGDVPRVDGQLAVARAFGDKSLKIHLSSDPDIRDENIDHETEFILFASDGVWKVFEISSHVIIRVMSNQEAVDLIKSIKDPQAAAKELIEEAVSKQSTDDISCIVVRFQ

SEQ ID NO: 33 - protein, 488 aa, Arabidopsis thaliana
MGDEPLLQKVKIQEDIESVPLLQKVKIQEDIESVKGIRVNNDGEEDGPVTLILLFTTFTALCGTFSYGTAAGFTSPAQTGIMAGLNLSLAEFSFFGAVLTIGGLVGAAMSGKLADVFGRRGALGVSNSFCMAGWLMIAFSQATWSLDIGRLFLGVAAGVASYVVPVYIVEIAPKKVRGTFSAINSLVMCASVAVTYLLGSVISWQKLALISTVPCVFEFVGLFFIPESPRWLSRNGRVKESEVSLQRLRGNNTDITKEAAEIKKYMDNLQEFKEDGFFDLFNPRYSRVVTVGIGLLVLQQLGGLSGYTFYLSSIFKKSGFPNNVGVMMASVVQSVTSVLGIVIVDKYGRRSLLTVATIMMCLGSLITGLSFLFQSYGLLEHYTPISTFMGVLVFLTSITIGIGGIPWVMISEMTPINIKGSAGTLCNLTSWSSNWFVSYTFNFLFQWSSSGVFFIYTMISGVGILFVMKMVPETRGRSLEEIQAAITR

SEQ ID NO: 34 - protein, 457 aa, Arabidopsis thaliana
MAEEGLLLPASSTSSSSSLLSEISNACTRPFVLAFIVGSCGAFAFGCIYSLFGSILTVGLILGALICGKLTDLVGRVKTIWITNILFVIGWFAIAFAKGVWLLDLGRLLQGISIGISVYLGPVYITEIAPRNLRGAASSFAQLFAGVGISVFYALGTIVAWRNLAILGCIPSLMVLPLLFFIPESPRWLAKVGREMEVEAVLLSLRGEKSDVSDEAAEILEYTEHVKQQQDIDDRGFFKLFQRKYAFSLTIGVVLIALPQLGGLNGYSFYTDSIFISTGVSSDFGFISTSVVQMFGGILGTVLVDVSGRRTLLLVSQAGMFLGCLTTAISFFLKENHCWETGTPVLALFSVMVYFGSYGSGMGSIPWIIASEIYPVDVKGAAGTMCNLVSSISAWLVAYSFSYLLQWSSTGTFLMFATVAGLGFVFIAKLVPETKGKSLEEIQSLFTDSPPQDSTIF

SEQ ID NO: 35 - protein, 729 aa, Arabidopsis thaliana
MSGAVLVAIAAAVGNLLQGWDNATIAGAVLYIKKEFNLESNPSVEGLIVAMSLIGATLITTCSGGVADWLGRRPMLILSSILYFVGSLVMLWSPNVYVLLLGRLLDGFGVGLVVTLVPIYISETAPPEIRGLLNTLPQFTGSGGMFLSYCMVFGMSLMPSPSWRLMLGVLFIPSLVFFFLTVFFLPESPRWLVSKGRMLEAKRVLQRLRGREDVSGEMALLVEGLGIGGETTIEEYIIGPADEVTDDHDIAVDKDQIKLYGAEEGLSWVARPVKGGSTMSVLSRHGSTMSRRQGSLIDPLVTLFGSVHEKMPDTGSMRSALFPHFGSMFSVGGNQPRHEDWDEENLVGEGEDYPSDHGDDSEDDLHSPLISRQTTSMEKDMPHTAHGTLSTFRHGSQVQGAQGEGAGSMGIGGGWQVAWKWTEREDESGQKEEGFPGSRRGSIVSLPGGDGTGEADFVQASALVSQPALYSKDLLKEHTIGPAMVHPSETTKGSIWHDLHDPGVKRALVVGVGLQILQQFSGINGVLYYTPQILEQAGVGILLSNMGISSSSASLLISALTTFVMLPAIAVAMRLMDLSGRRTLLLTTIPILIASLLVLVISNLVHMNSIVHAVLSTVSVVLYFCFFVMGFGPAPNILCSEIFPTRVRGICIAICALTFWICDIIVTYSLPVLLKSIGLAGVFGMYAIVCCISWVFVFIKVPETKGMPLEVITEFFSVGARQAEAAKNE

SEQ ID NO: 36 - protein, 493 aa, Arabidopsis thaliana
MADQISGEKPAGVNRFALQCAIVASIVSIIFGYDTGVMSGAMVFIEEDLKTNDVQIEVLTGILNLCALVGSLLAGRTSDIIGRRYTIVLASILFMLGSILMGWGPNYPVLLSGRCTAGLGVGFALMVAPVYSAEIATASHRGLLASLPHLCISIGILLGYIVNYFFSKLPMHIGWRLMLGIAAVPSLVLAFGILKMPESPRWLIMQGRLKEGKEILELVSNSPEEAELRFQDIKAAAGIDPKCVDDVVKMEGKKTHGEGVWKELILRPTPAVRRVLLTALGIHFFQHASGIEAVLLYGPRIFKKAGITTKDKLFLVTIGVGIMKTTFIFTATLLLDKVGRRKLLLTSVGGMVIALTMLGFGLTMAQNAGGKLAWALVLSIVAAYSFVAFFSIGLGPITWVYSSEVFPLKLRAQGASLGVAVNRVMNATVSMSFLSLTSAITTGGAFFMFAGVAAVAWNFFFFLLPETKGKSLEEIEALFQRDGDKVRGENGAA

SEQ ID NO: 37 - protein, 560 aa, Arabidopsis thaliana
MQSSTYAVKGNAAFAFQRRTFSSDRSTTSTGIRFAGYKSLATTGPLYCSGSEAMGATLARADNGIQSVMSFSSVKARSVRAQASSDGDEEEAIPLRSEGKSSGTVLPFVGVACLGAILFGYHLGVVNGALEYLAKDLGIAENTVLQGKYMMIHFFTPPVNGWIVSSLLAGATVGSFTGGALADKFGRTRTFQLDAIPLAIGAFLCATAQSVQTMIVGRLLAGIGIGISSAIVPLYISEISPTEIRGALGSVNQLFICIGILAALIAGLPLAANPLWWRTMFGVAVIPSVLLAIGMAFSPESPRWLVQQGKVSEAEKAIKTLYGKERVVELVRDLSASGQGSSEPEAGWFDLFSSRYWKVVSVGAALFLFQQLAGINAVVYYSTSVFRSAGIQSDVAASALVGASNVFGTAVASSLMDKMGRKSLLLTSFGGMALSMLLLSLSFTWKALAAYSGTLAVVGTVLYVLSFSLGAGPVPALLLPEIFASRIRAKAVALSLGMHWISNFVIGLYFLSVVTKFGISSVYLGFAGVCVLAVLYIAGNVVETKGRSLEEIELALTSGA

SEQ ID NO: 38 - protein, 440 aa, Arabidopsis thaliana
MALDPEQQQPISSVSRESSGEISPEREPLIKENHVPENYSVVAAILPFLFPALGGLLYGYEIGATSCATISLQEPMTLLSYYAVPFSAVAFIKWNFMTSGSLYGALFGSIVAFTIADVIGRRKELILAALLYLVGALVTALAPTYSVLIIGRVIYGVSVGLAMHAAPMYIAETAPSPIRGQLVSLKEFFIVLGMVGGYGIGSLTVNVHSGWRYMYATSVPLAVIMGIGMWWLPASPRWLLLRVIQGKGNVENQREAAIKSLCCLRGPAFVDSAAEQVNEILAELTFVGEDKEVTFGELFQGKCLKALIIGGGLVLFQQLIMTGVAVVVIDRLGRRPLLLGGVGGMRLTSCCCSCTAALCGLLPEIFPLKLRGRGLSLAVLVNFGANALVTFAFSPLKELLGAGILFCGFGVICVLSLVFIFFIVPETKGLTLEEIEAKCL

SEQ ID NO: 39 - protein, 475 aa, Arabidopsis thaliana
MAIREIKDVERGEIVNKVEDLGKPFLTHEDDEKESENNESYLMVLFSTFVAVCGSFEFGSCVGYSAPTQSSIRQDLNLSLAEFSMFGSILTIGAMLGAVMSGKISDFSGRKGAMRTSACFCITGWLAVFFTKGALLLDVGRFFTGYGIGVFSYVVPVYIAEISPKNLRGGLTTLNQLMIVIGSSVSFLIGSLISWKTLALTGLAPCIVLLFGLCFIPESPRWLAKAGHEKEFRVALQKLRGKDADITNEADGIQVSIQALEILPKARIQDLVSKKYGRSVIIGVSLMVFQQFVGINGIGFYASETFVKAGFTSGKLGTIAIACVQVPITVLGTILIDKSGRRPLIMISAGGIFLGCILTGTSFLLKGQSLLLEWVPSLAVGGVLIYVAAFSIGMGPVPWVIMSEGIAGSLVVLVNWSGAWAVSYTFNFLMSWSSPGTFYLYSAFAAATIIFVAKMVPETKGKTLEEIQACIRRET

SEQ ID NO: 40 - protein, 469 aa, Arabidopsis thaliana
MEEGRSIEEGLLQLKNKNDDSECRITACVILSTFVAVCGSFSFGVATGYTSGAETGVMKDLDLSIAQFSAFGSFATLGAAIGALFCGNLAMVIGRRGTMWVSDFLCITGWLSIAFAKEVVLLNFGRIISGIGFGLTSYVVPVYIAEITPKHVRGTFTFSNQLLQNAGLAMIYFCGNFITWRTLALLGALPCFIQVIGLFFVPESPRWLAKVGSDKELENSLFRLRGRDADISREASEIQVMTKMVENDSKSSFSDLFQRKYRYTLVVGIGLMLIQQFSGSAAVISYASTIFRKAGFSVAIGTTMLGIFVIPKAMIGLILVDKWGRRPLLMTSAFGMSMTCMLLGVAFTLQKMQLLSELTPILSFICVMMYIATYAIGLGGLPWVIMSEIFPINIKVTAGSIVTLVSFSSSSIVTYAFNFLFEWSTQGIMFFFFLISILYNFGYINYLVINKKSYNLSFFFVYRIEIIND

(19) United States
(12) Patent Application Publication
(10) Pub. No.: US 2005/0125159 A1
(43) Pub. Date: Jun. 9, 2005

(54) Methods for analyzing biological elements

(76) Inventors: Joshua C. Stein, Acton, MA (US); Yongwei Cao, Chesterfield, MO (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/213,974

(22) Filed: Aug. 6, 2002

Related U.S. Application Data
(60) Provisional application No. 60/325,537, filed on Oct. 1, 2001.

(51) Int. Cl. G06F 19/00; G01N 33/48; G01N 33/50

(52) U.S. Cl. 702/20

(57) ABSTRACT

The present invention is in the field of bioinformatics, particularly as it pertains to determining the associations of biological elements. More specifically, the present invention relates to the determination of associations among a set of biological elements using methods capable of generating and sorting clusters of biological elements.

### CROSS-REFERENCE TO RELATED APPLICATION

This application claims priority under 35 U.S.C. §119(e) of U.S. Provisional Application No. 60/325,537 filed Oct. 1, 2001, the disclosure of which application is incorporated herein by reference in its entirety.

### FIELD OF THE INVENTION

The present invention is in the field of bioinformatics, particularly as it pertains to determining the associations of biological elements. More specifically, the present invention relates to the determination of associations among a set of biological elements using methods capable of generating and sorting clusters of biological elements.

### BACKGROUND OF THE INVENTION

Recent advances across the spectrum of the biological sciences have allowed researchers to compile large amounts of biological data from a myriad of organisms. For example, advances in genome sequencing and gene prediction have resulted in a rapid increase in the amount of raw sequence data stored in both nucleic acid and protein sequence databases. The rapid accumulation of these data, however, has not been accompanied by an equivalently rapid understanding of the complex biological relationships that exist among the biological elements represented by that accumulated data.

Various methods for determining relationships among the biological elements in databases have been reported (see, for example Chervitz et al., Science, 282:2022-2028 (1998); Rubin et al., Science, 287:2204-2215 (2000); Venter et al., Science, 291:1304-1351 (2001); and, Tatusov et al., Science, 278:631-637 (1997)).

Some reported methods have attempted to classify groups of genes or proteins by level of sequence similarity. This approach, although simple and direct, can lead to incomplete or undesirable groupings. As shown in FIG. 1, for example, conventional grouping methods that attempt to use only a direct sequence similarity comparison can fail to detect relationships among biological elements in a set. In the schematic example shown in FIG. 1, if a sequence similarity comparison is performed for sequence A against all other members of a set at a defined relatedness of 30% or greater, then sequence B will be returned as sufficiently related, but sequence C will not. One obvious shortcoming of this conventional grouping strategy is seen when sequence B is compared to sequence C and it is recognized that the two are as similar as sequence B is to sequence A. This results in a grouping that entirely neglects both the relationship between sequence B and sequence C as well as any potential relationship between sequence A and sequence C that is implicated by the relationship between sequence B and sequence C. As a result, conventional grouping methods can yield results that group sequences without any indication of the relatedness of members of any cluster produced other than the single grouping parameter used to perform the grouping.

A further disadvantage of conventional grouping methods is seen, for example, when databases comprising large numbers of multi-domain protein sequences are searched using the above methodology. A search performed at a low level defined relatedness will tend to return large numbers of protein sequences that are unrelated except for a domain that is common to many different types of protein. For example, leucine rich repeat (LRR) regions occur in many proteins, and can cause the undesirable grouping of proteins that are otherwise unrelated. In response, an investigator can, of course, increase the defined relatedness and rerun the search, but such an approach can lead to large sets of data that are difficult to analyze.

What is needed in the art are methods to rapidly cluster a set of biological elements into related clusters at several defined levels of relatedness and to then sort the resulting clusters for efficient and accurate analysis.

### SUMMARY OF THE INVENTION

The present invention includes and provides a method of analyzing a set of biological elements comprising: a) performing a comparison of the set; b) applying a transitive clustering algorithm at a defined relatedness to the set using results of the comparison to produce one or more clusters; c) repeating step b) one or more times at different levels of relatedness; and d) sorting the biological elements based on the clusters.

The present invention includes and provides a method of analyzing a set of biological elements comprising: a) performing a comparison of the set; b) applying a transitive clustering algorithm at a defined relatedness to the set using results of the comparison to produce one or more clusters; c) repeating step b) one or more times at different levels of relatedness; d) sorting the biological elements based on the clusters; and, e) displaying results of the sorting.

The present invention includes and provides a program storage device readable by a machine, tangibly embodying a program of instructions executable by a machine to perform method steps to analyze a set of biological elements comprising: a) performing a comparison of the set; b) applying a transitive clustering algorithm at a defined relatedness to the set using results of the comparison to produce one or more clusters; c) repeating step b) one or more times at different levels of relatedness; and d) sorting the biological elements based on the clusters.

Description of the Sequences

TABLE 1

| SEQ ID NO: | Identifying Name | Description |
|---|---|---|
| 1 | F6F3.26#At1g01280#68170.m00027 | cytochrome P450, putative |
| 2 | F6F3.15#At1g01340#68170.m00033 | cyclic nucleotide and calmodulin- |
| | | regulated ion channel, putative |
| 3 | YUP8H12.23#At1g05160#68170.m00422 | putative cytochrome P450 |
| 4 | F22G5.17#At1g07430#68170.m00628 | protein phosphatase 2C, putative |
| 5 | T12M4.13#At1g09160#68170.m00803 | putative protein phosphatase 2C |
| 6 | F25C20.25#At1g11600#68170.m01054 | putative cytochrome P450 |
| 7 | F3F19.9#At1g13070#68170.m01176 | putative cytochrome P450 |
| | | monooxygenase |
| 8 | F3O9.3#At1g16220#68170.m01483 | putative protein phosphatase 2C |
| 9 | F3O9.21#At1g16410#68170.m01502 | putative cytochrome P450 |
| 10 | F20D23.24#At1g17060#68170.m01583 | putative cytochrome P450 |
| 11 | F14P1.46#At1g19780#68170.m01817 | cyclic nucleotide and calmodulin- |
| | | regulated ion channel, putative |
| 12 | F21J9.120#At1g24540#68170.m02299 | putative cytochrome P450 |
| 13 | F21J9.40#At1g24620#68170.m02307 | putative calmodulin |
| 14 | F27G20.1#At1g32250#68170.m02939 | calmodulin, putative |
| 15 | F14M2.11#At1g33730#68170.m03090 | cytochrome P450, putative |
| 16 | F12M16.28#At1g53390#68170.m04287 | putative ABC transporter |
| | | gb\|AAD31586.1 |
| 17 | F23N19.25#At1g62820#68170.m05027 | calmodulin, putative |
| 18 | F1N19.12#At1g64550#68170.m05196 | ABC transporter protein, putative |
| 19 | T27F4.15#At1g66400#68170.m05380 | calmodulin-related protein |
| 20 | T27F4.1#At1g66410#68170.m05381 | calmodulin-4 |
| 21 | T4O24.9#At1g66950#68170.m05428 | ABC transporter, putative |
| 22 | T23K23.21#At1g67940#68170.m05546 | putative ABC transporter |
| 23 | F5A18.21#At1g70610#68170.m05805 | putative ABC transporter |
| 24 | F3I17.2#At1g71330#68170.m05855 | putative ABC transporter |
| 25 | F17M19.11#At1g71960#68170.m05894 | putative ABC transporter |
| 26 | F28P22.4#At1g72770#68170.m05953 | protein phosphatase 2C (AtP2C-HA) |
| 27 | F25P22.4#At1g73630#68170.m06060 | putative calmodulin |
| 28 | F28K19.17#At1g77960#68170.m06480 | similar to phosphate ABC |
| | | transporter, permease protein (pstC) |
| | | gi\|2688114 |
| 29 | T11I11.14#At1g78200#68170.m06504 | putative protein phosphatase 2C |
| 30 | T1O16.14#At2g14270#51595.m09604 | putative protein phosphatase 2C |
| 31 | T2G17.15#At2g20050#51595.m10178 | putative protein phosphatase 2C |
| 32 | F23N11.5#At2g20630#51595.m10236 | putative protein phosphatase 2C |
| 33 | MQC12.22#At3g20460#68173.m01984 | sugar transporter, putative |
| 34 | T4B21.9#At4g04760#68164.m00476 | putative sugar transporter |
| 35 | F23E12.140#At4g35300#68164.m03354 | putative sugar transporter protein |
| 36 | C7A10.690#At4g36670#68164.m03485 | sugar transporter like protein |
| 37 | T21H19.70#At5g16150#68172.m01416 | sugar transporter-like protein |
| 38 | F2K13.160#At5g17010#68172.m01503 | sugar transporter-like protein |
| 39 | F17K4.90#At5g18840#68172.m01689 | sugar transporter-like protein |
| 40 | F21A20.60#At5g27350#68172.m02435 | sugar transporter-like protein |

Table Headings:
“SEQ ID NO:” is the number of the sequence for the purposes of the sequence listing.
“Identifying Name” is a name assigned to the sequence.
“Description” is a public annotation provided for the sequence, and may include a gi number or GenBank identifier.

### DESCRIPTION OF THE FIGURES

FIG. 1 is a schematic representation of a conventional sequence similarity grouping method.

FIG. 2 is a flow diagram of one embodiment of the present invention.

FIGS. 3a through 3e are a schematic representation of the operation of one transitive clustering algorithm that can be used in the present invention.

FIG. 4 is a schematic representation of the operation of one transitive clustering algorithm that can be used in the present invention.

FIGS. 5a through 5c are tables representing one embodiment of sorting of biological elements.

FIG. 6 is a schematic illustration of one embodiment of a computer system that is capable of implementing methods of the present invention.

FIG. 7 is a schematic illustration of another embodiment of a computer system that is capable of implementing methods of the present invention.

FIGS. 8a and 8b are clustergrams representing the output of Example 4.

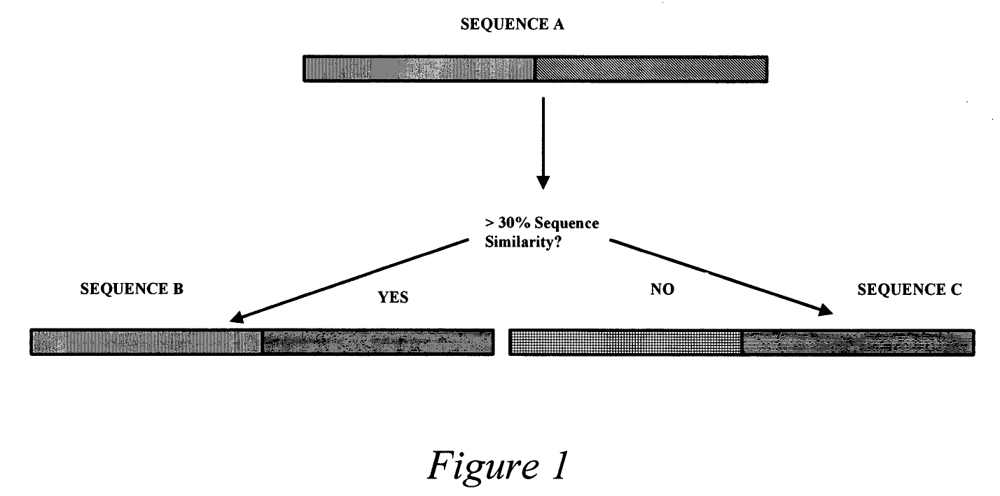

### DETAILED DESCRIPTION OF THE INVENTION

Described herein are methods for determining the associations among a set of biological elements. Also described herein are program storage devices readable by a machine, tangibly embodying a program of instructions executable by a machine to perform the method steps of the present invention. The present invention allows for the efficient clustering of biological elements within a set at varying levels of relatedness, and the subsequent sorting of the generated clusters in a manner that allows for convenient visualization of biological element relatedness.

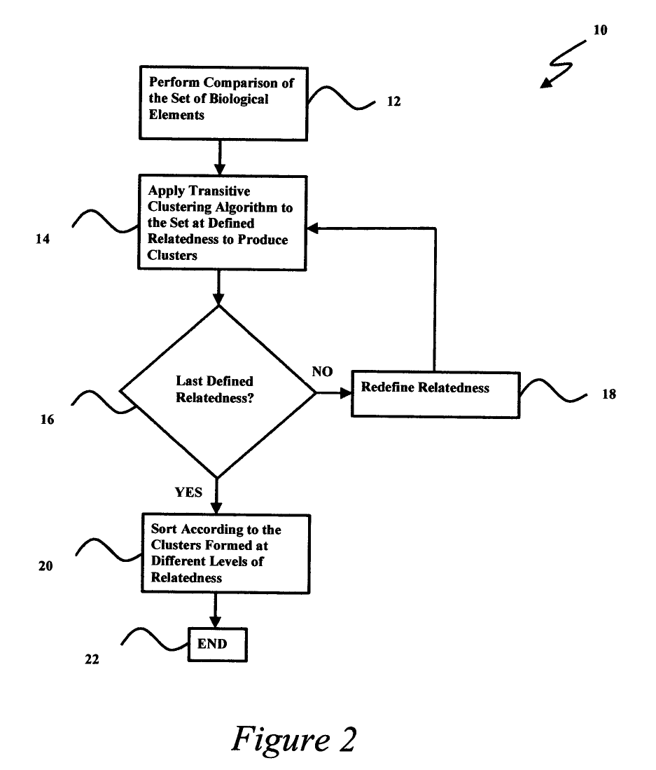

One embodiment of a method of the present invention is shown in FIG. 2 generally at 10. As shown in FIG. 2, in step 12 a comparison of a set of biological elements is performed. This comparison yields a set of data that associates the biological elements of the set. In step 14, information from the data set is used by a transitive clustering algorithm, which clusters the biological elements of the set at a defined relatedness. In step 16, it is determined if the last defined relatedness has been reached. If not, then flow continues to step 18, where the relatedness is redefined, and flow proceeds to step 14, where the transitive clustering algorithm clusters the biological elements of the set at the newly defined relatedness. When the last defined relatedness is reached in step 16, flow proceeds to step 20, where the clusters produced by the transitive clustering algorithm in 14 are sorted. Flow then ends in step 22.

As used herein, “performing a comparison” of a set of biological elements means using a method of comparing biological elements to produce a data set that represents relationships among the biological elements. As used herein, a “biological element” is any physical entity or component of a biological system or anything that interacts or affects a biological system or any other component of a biological system that can be quantified, and a “set” of biological elements is any grouping of biological elements greater than one. A biological element can be, for example and without limitation, an atomic particle, an atom, a molecule, a compound, or combination thereof, including cellular organisms. A biological system can be any living organism, virus, cell, or components derived therefrom. In a preferred embodiment, biological elements comprise amino acid sequences. In another preferred embodiment, biological elements comprise nucleic acid sequences, e.g. genomic DNA sequences, RNA sequences, or cDNA sequences. In a further preferred embodiment, biological elements comprise cDNA sequences. In a further preferred embodiment, biological elements comprise enzymes. In another preferred embodiment, biological elements comprise expression profiles (TxP). In another embodiment, sets can comprise a single type of biological element, such as a protein sequence database, or multiple types of biological elements, such as cDNA sequences and genomic sequences.

As used herein, a “set of biological elements” can be any form of representation of biological elements that can be inputted into the method of comparison being used. Representations include numerical and symbolic forms, such as numbers and letters. In a preferred embodiment, one letter representations of amino acid or nucleic acid sequences are used. In a preferred embodiment, the set of biological elements comprises amino acid sequences. In another preferred embodiment, the set of biological elements comprises nucleic acid sequences.

Any method for performing a comparison that produces a data set that represents relationships among the biological elements of the set can be used. In a preferred embodiment, the method for performing a comparison is the execution of a computer program designed to compare biological elements. In a preferred embodiment, the comparison is a BLAST comparison. In another preferred embodiment, the comparison is a BLASTP comparison. In such programs, the output of the comparison is potentially not limited to a single measure of relatedness. For example, sequence comparisons generated by BLAST programs can concurrently produce different statistical measures of sequence relatedness, such as percent identity, percent similarity, e-value, bit score, and fraction of query and hit. In yet another preferred embodiment the output from a BLAST comparison is inputted into blastpl, which parses the BLAST output.

Any statistical measure that results in a value that represents a relationship between biological elements of a set can be used for a given method of comparison. In another preferred embodiment, statistical measures that incorporate more than one type of sequence relatedness measure can be used. For example, both e-value and fraction of query and hit can be mathematically combined into a single result for purposes of the comparison. In another embodiment, one type of sequence relatedness measure can be used on a group of biological elements for the purpose of removing elements that lack a desired level of relatedness with any other members of the group before any comparison for the purposes of grouping is done. Thereafter, the same or a different measure of relatedness can be used for the clustering. As used herein, “fraction of query and hit” is determined as follows: for any two sequences in a set, for example A and B, the number of common “hits” is divided by the total number of noncommon hits for A and B together, and the result is converted to a percentage. For example, if A had 20 hits, B had 10 hits, and 5 hits on A and B were the same, then fraction of query and hit would yield a result of 5/(10+20−5)=0.2 or 20%.

In a preferred embodiment, the comparison performed is an all-versus-all comparison. An all-versus-all comparison, as used herein, is a comparison whereby each member of a set is compared to every other member of the set. The results of an all-versus-all comparison can be, for example, a data set with each member of the set having associated values of relatedness to every other member of the set. In a simple four member set, for example, an all-versus-all comparison could entail, for example, comparing 1 to each of 2, 3, and 4, and then comparing 2 to each of 3 and 4, and then comparing 3 to 4, whereby the relatedness, as determined by the statistical method of the comparison, of each member to every other member is thereby known.

After the comparison of biological elements is performed, an algorithm is applied to the comparison results, which can be, for example, a data set (various algorithms have been reported, for example by Kriventseva et al., Nucleic Acids Research, Vol. 29(1):33-36 (2001) and Gerstein, Yale University web site (bioinfo.mmb.yale.edu/e-print/transcmp-bioinfo-preprint.htm)). The algorithm can be any algorithm that is capable of clustering the biological elements of the set into related clusters of related biological elements based upon the results of the comparison (see, for example, Johnson and Wichern, Applied Multivariate Statistical Analysis, fourth edition (1998), pages 726-760, Prentice-Hall, Inc. New Jersey; and, Cawsey, The Essence of Artificial Intelligence, first edition (1998), pages 68-95, Prentice-Hall PTR). In a preferred embodiment, the algorithm is a transitive clustering algorithm. In a further preferred embodiment, the algorithm is the transitive clustering algorithm described below in example 1 having the file name script yc_cluster_inc100.pl.

As used herein, “applying” an algorithm means inputting data into the algorithm, executing the steps of the algorithm, and outputting results from the algorithm. As used herein, a “transitive clustering algorithm” is any algorithm that can be applied to the results of the comparison and output a cluster of biological elements of the set where each biological element of the cluster is related to at least one other biological element of the cluster with at least a defined relatedness, and where every biological element of the cluster is not related to any biological element of the set that is not in the cluster at or above the level of the defined relatedness. In a preferred embodiment, a transitive clustering algorithm of the present invention is capable of outputting one or more clusters, where for each cluster thereby outputted, each biological element of the cluster is related to at least one other biological element of the cluster with at least a defined relatedness, and where every biological element of the cluster is not related to any biological element of the set that is not in the cluster at or above the level of the defined relatedness. In another preferred embodiment, a transitive clustering algorithm of the present invention, when applied to a set of biological elements, is capable of producing one or more clusters where each biological element of the set is in only one cluster, and where, for each cluster, each biological element of the cluster is related to at least one other biological element of the cluster with at least a defined relatedness, and where every biological element of the cluster is not related to any biological element of the set that is not in the cluster at or above the level of the defined relatedness.

As used herein, a “defined relatedness” is a threshold value below which two biological elements will not be considered sufficiently related to cluster together based on their direct comparison. Of course, as described above and discussed in the example below, two biological elements that do not reach the defined relatedness level between themselves can still be clustered together if they are sufficiently related to one or more other biological elements—that is, if they are sufficiently transitively related. The defined relatedness can be set at any level for any single loop through the algorithm, according to the intent of the investigator. The defined relatedness will be a value that reflects the statistical comparison that is performed. For example, if percent identity is used as the method of comparison among a set of sequences, then the defined relatedness used in the algorithm will be a value between zero and one hundred, inclusive. In a preferred embodiment, the defined relatedness is a value derived from a member of the group consisting of percent identity, percent similarity, e-value, bit score, and fraction of query and hit. In a more preferred embodiment, the defined relatedness is a value derived from fraction of query and hit.

As shown in FIG. 2 and as described herein, more than one level of defined relatedness is used in the present invention. In a preferred embodiment, the defined relatedness is ramped upward from an initial low value to a final high value, thereby allowing an even segregation of clusters for later sorting. For example, the initial value of the defined relatedness for an algorithm that is clustering the results of a percent identity comparison can be set at 20, with the relatedness redefined each subsequent loop through the algorithm at a value of 2 greater than the previous loop until a maximum value of 100 is reached. In this manner the transitive clustering algorithm produces a group of clusters at the 20 percent identity level of relatedness, a second group of clusters at the 22 percent identity level of relatedness, and so on, until the final group of clusters is produced at the 100 percent identity level of relatedness. Any number of levels of defined relatedness can be used, and the choice of which levels to use and what the gradation between levels should be will typically depend on the size and nature of the set of biological elements under study. Although the algorithm can be designed to loop through the various levels of defined relatedness in any order, in a preferred embodiment the defined relatedness is increased during each loop. In a preferred embodiment, 100 levels of defined relatedness are used, varying in 0.01 increments from a fraction of query and hit value of 0.01 to 1.00. In another preferred embodiment, at least 10 levels of defined relatedness are used, and more preferably, at least 20, 30, 40, 50, 60, 70, 80, 90, or 100 levels of defined relatedness are used.

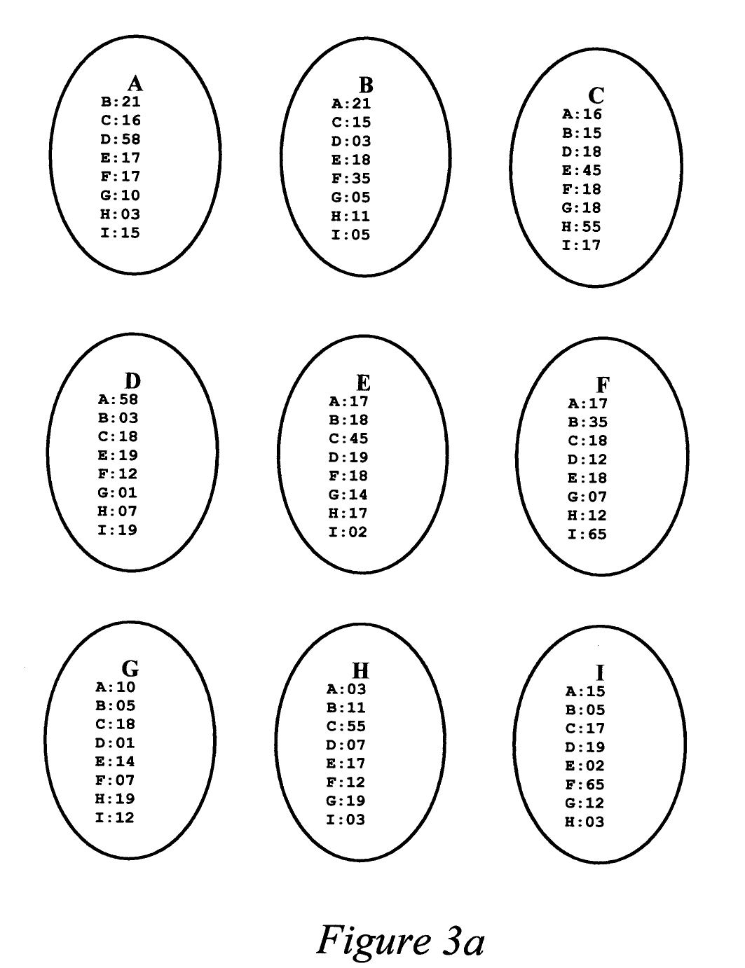

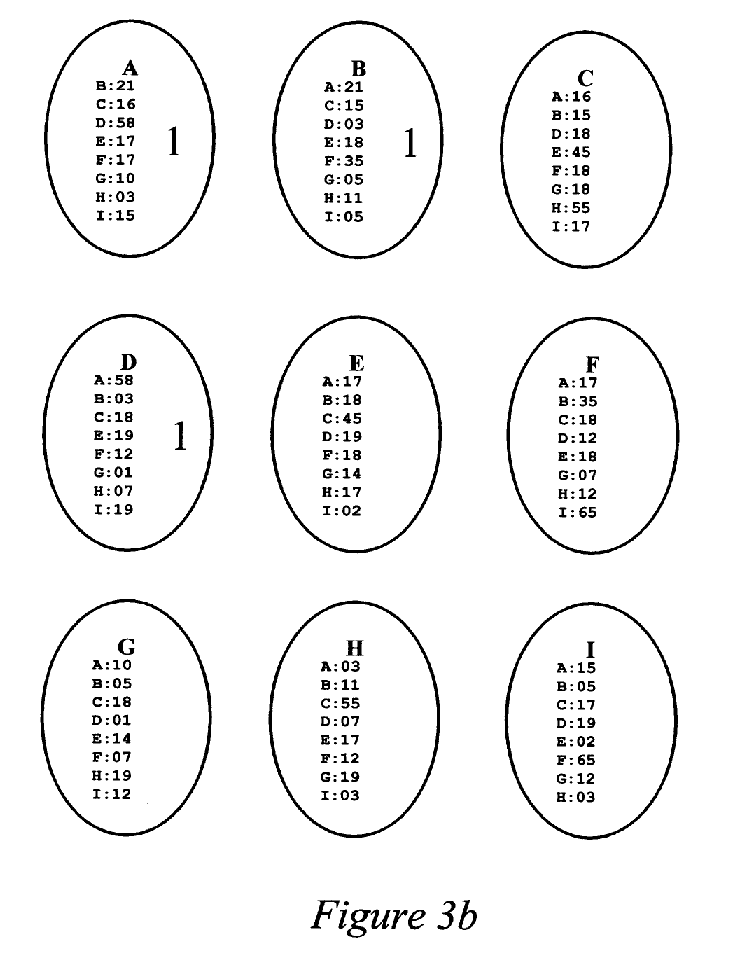

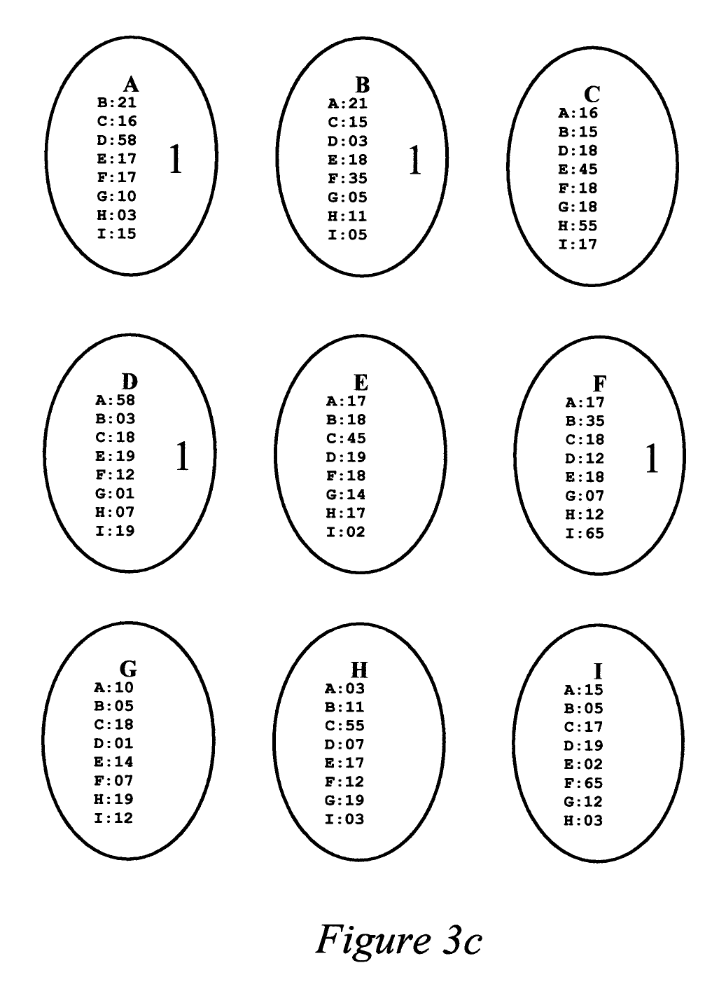

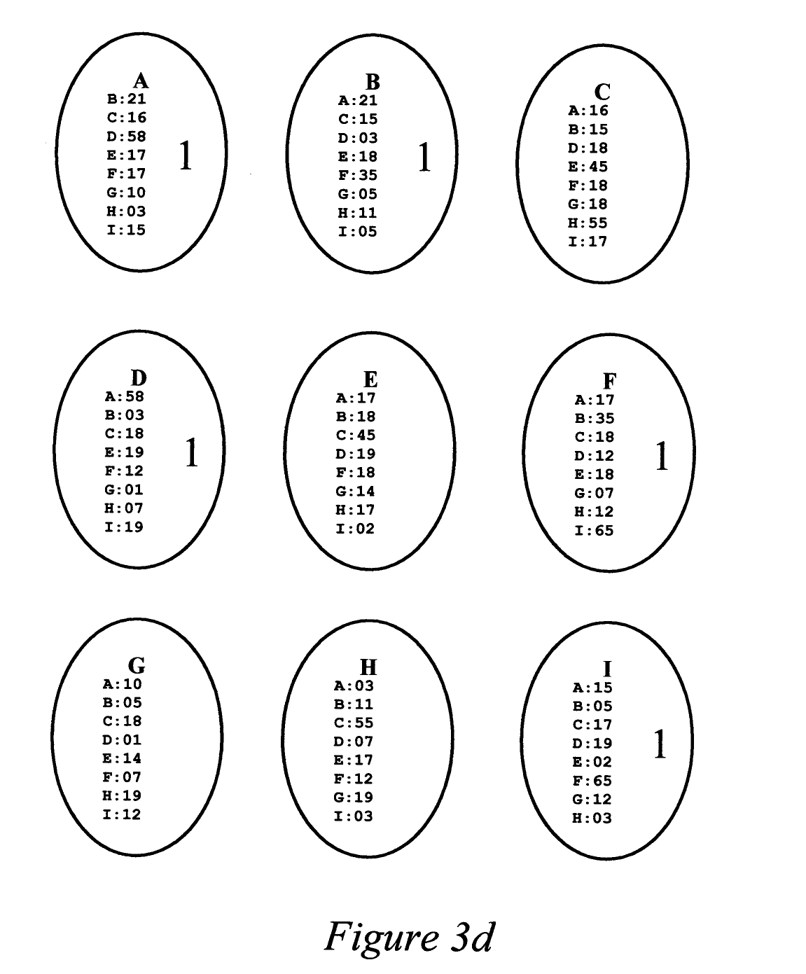

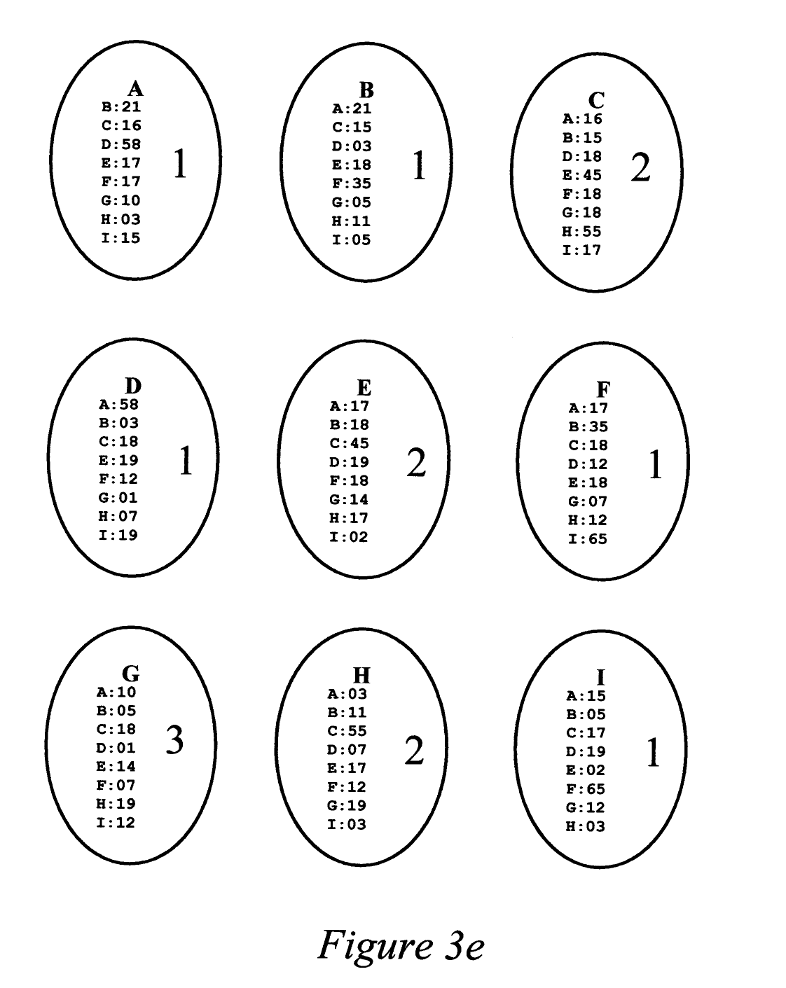

FIGS. 3a through 3e represent an illustrative example of a transitive clustering algorithm that can be used to cluster biological elements of the present invention. In this example, a single clustering at a level of relatedness of greater than 20% is shown. FIG. 3a represents an example of a set of biological elements that have been compared. In FIG. 3a, each biological element is represented as an oval with an identifying letter at the top. FIG. 3a represents a set of biological elements where the set comprises nine biological elements lettered A, B, C, D, E, F, G, H, and I. For exemplary purposes, an all-versus-all comparison has already been performed on the set, and the results are represented by the data within the oval of each biological element. For example, biological element A has a relatedness of 21% with biological element B, a relatedness of 16% with biological element C, a relatedness of 58% with D, and so on. As shown in FIG. 3a, the relatedness of a given biological element to each other biological element of the set is shown in the oval for that given biological element. In this embodiment, a transitive clustering algorithm of the present invention begins the formation of a first cluster by associating a first biological element of the set with any other biological elements of the set that have greater than a defined relatedness to the first biological element. Any biological element can be used as the first biological element. In this example, biological element A is used as the first biological element. The different levels of relatedness of biological element A to the other biological elements of the set shown within the oval representation of biological element A are examined for any relatedness of at least 20%—that is, at least at the level of the defined relatedness for this clustering, and it is found that biological elements B (21%) and D (58%) have at least the defined relatedness to biological element A. After this step, as shown by the large numeral in the right side of the ovals of the biological elements in FIG. 3b, biological elements A, B, and D have been associated in a first cluster (cluster 1). At this stage, all of the biological elements of the set that have at least the defined relatedness of 20% to biological element A are associated in the first cluster, but it is not certain that all of the biological elements of the set that have equal to or greater than the defined relatedness with biological elements B or D have been associated with the first cluster. The next step is therefore to associate in the first cluster any biological elements of the set that are not already in the first cluster that have at least the defined relatedness to any member of the first cluster. In this example, the levels of relatedness shown in the ovals for biological element B and for biological element D are examined, and it is determined that biological element F (35% related to B) has at least the defined relatedness to biological element B. Biological element F is therefore associated with the other biological elements in the first cluster, as shown in FIG. 3c.

The step is repeated, and it is determined that biological element I has at least the defined relatedness to biological element F, and so biological element I is associated with the first cluster, as shown in FIG. 3d. At this stage, no biological element that has not already been associated with the first cluster has at least the defined relatedness to any of the biological elements of the first cluster, and so the first cluster is complete.

The entire clustering process described above that started with biological element A can now be repeated for the biological elements of the set that have not been associated with the first cluster to arrive at the complete clustering shown in FIG. 3e. As shown in FIG. 3e, biological elements C, E, and H have been associated in a second cluster, and biological element G is associated with a third cluster.

At this stage, each biological element of the set has been associated in one of the three clusters formed at a defined relatedness of 20%. To further analyze the set, the above-described method of clustering can be applied to the set of biological elements at a defined relatedness that is greater than the one previously used. For example, the method can be performed defined relatedness of, for example, 30%. The first cluster, comprising biological elements A, B, D, F, and I, will then be further clustered into cluster 4, comprising A and D, and cluster 5, comprising B, F, and I. If the clustering algorithm is applied at a higher level of defined relatedness but a particular cluster loses no members (that is, is not subdivided into two or more smaller clusters), then, for the purposes of cluster identification, the number of the cluster can remain the same for both the lower and higher level of defined relatedness. For example, in the above example biological element G will be remain in the same cluster regardless of how many more loops at higher levels of defined relatedness are performed, because the cluster of one biological element can not be subdivided into two or more smaller clusters.

In general, as determined by the relatedness of the biological elements of a set of n biological elements and the defined level of relatedness used, a first cluster will comprise anywhere from 1 to n biological elements, inclusive. Further, depending on the relatedness of the biological elements of a set of n biological elements and the defined level of relatedness used, the number of clusters formed after each biological element of a set has been associated with a cluster is anywhere from 1 to n, inclusive. For example, if a set comprises 1,000 biological elements none of which are related to any other member at greater than a 10% level of relatedness, the above-described method applied at a defined level of relatedness of greater than 10% (e.g. 11%) will result in 1,000 clusters being formed, with each cluster containing a single biological element. Conversely, if a different set of 1,000 biological elements is used in which every biological element of the set is transitively related to every other biological element of the set at a level of 20% relatedness or more, the above-described method applied at a defined level of relatedness of 15% would yield a single cluster having 1,000 biological elements associated therewith. Of course, any number of clusters each with any number of biological elements is possible, depending upon the relatedness of the biological elements of the set and the defined relatedness chosen.

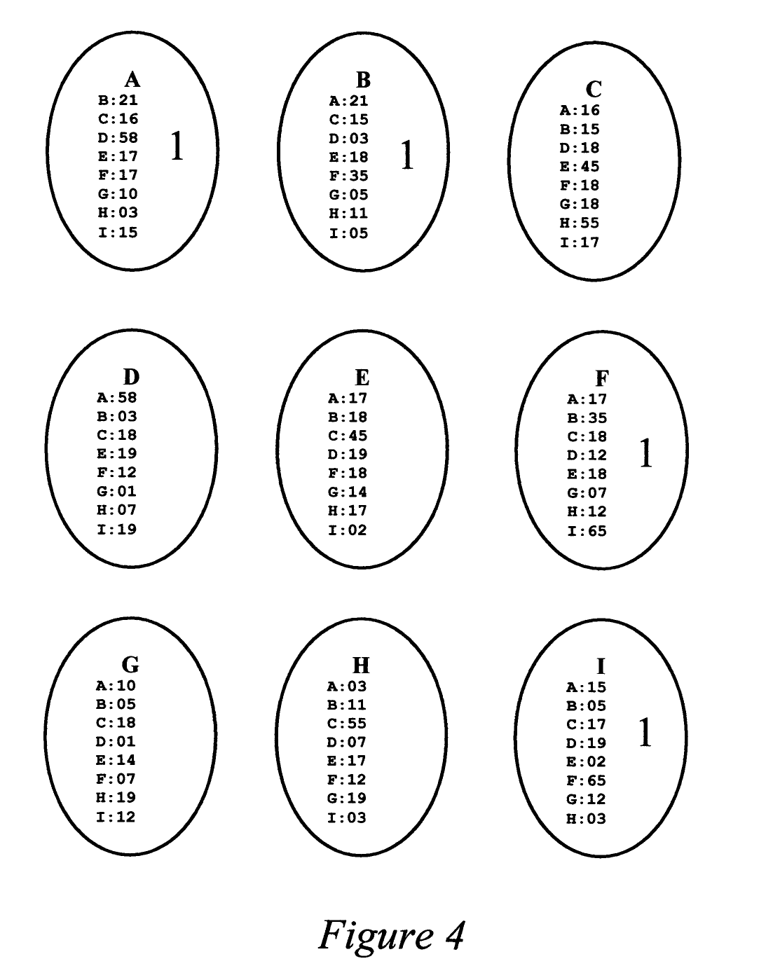

Having described one method of transitively clustering the biological elements of a set, another method will now be described. Taking the exemplary set of biological elements shown in FIG. 3a once again, a method of clustering biological elements within a set of biological elements is used wherein a first element of the set is examined for relatedness to the other biological elements of the set. Choosing a 20% defined relatedness again, the levels of relatedness shown in the oval of biological element A are examined until a biological element having at least the defined relatedness is found, which, in this example, is biological element B. If none had been found, then A would be associated in a first cluster by itself In this case, however, biological element B is associated with biological element A in a first cluster, and the levels of relatedness of biological element B to the other biological elements of the set are examined until a biological element that has at least the defined relatedness to biological element B is found. If none had been found, then flow would have returned to the levels of relatedness shown in the oval for biological element A for the element immediately after biological element B. In this case, however, biological element F is found to have at least 20% relatedness to biological element B, and so biological element F is associated in the first cluster, and flow proceeds to the biological elements and levels of relatedness shown in the oval representing biological element F. Again, each level of relatedness is examined until biological element I is determined to have at least the defined level of relatedness, at which time biological element I is associated in the first cluster and flow proceeds to the levels of relatedness shown in the oval representing biological element I. Examination of the levels of relatedness of biological element I to the other biological elements of the set reveals that none that are not already associated with the first cluster have at least the defined relatedness, and so flow proceeds back to the levels of relatedness shown in the oval for biological element F, but since no levels are given after the level of relatedness for biological element I, flow returns to the levels of relatedness of biological element B where the levels of relatedness for the biological elements after biological element F are examined. It is determined that no other biological elements have at least the defined level of relatedness to biological element B, and so flow returns to the levels of relatedness of biological element A directly after the level of relatedness to biological element B (21%). At this stage, the biological elements that have been associated in the first cluster are shown in FIG. 4. Each of biological elements B, F, and I have been examined and any biological elements with at least the defined relatedness to any of B, F, and I have been associated with the first cluster. The nested iteration process is repeated for the level of relatedness of each biological element shown in the oval for biological element A, and, in this manner, the first cluster shown in FIG. 3d is arrived at. Repetition of the process for the remaining biological elements leads to the clustering shown in FIG. 3e. It is understood that other embodiments for transitively clustering biological elements within a set of biological elements are within the spirit and scope of the present invention, and that that scope should not be limited by the embodiments described above.

After a clustering algorithm has been applied at more than one level of defined relatedness, the biological elements of the set can be sorted based on the results of the clustering. As used herein, “sorting” refers to organizing biological elements by reference identifier (such as a number or letter), by location or place in a database or table, or graphically, or any combinations of the foregoing. In a preferred embodiment, the sorting is hierarchical sorting. As used herein, “hierarchical sorting” is sorting that orders biological elements by cluster number, as described below.

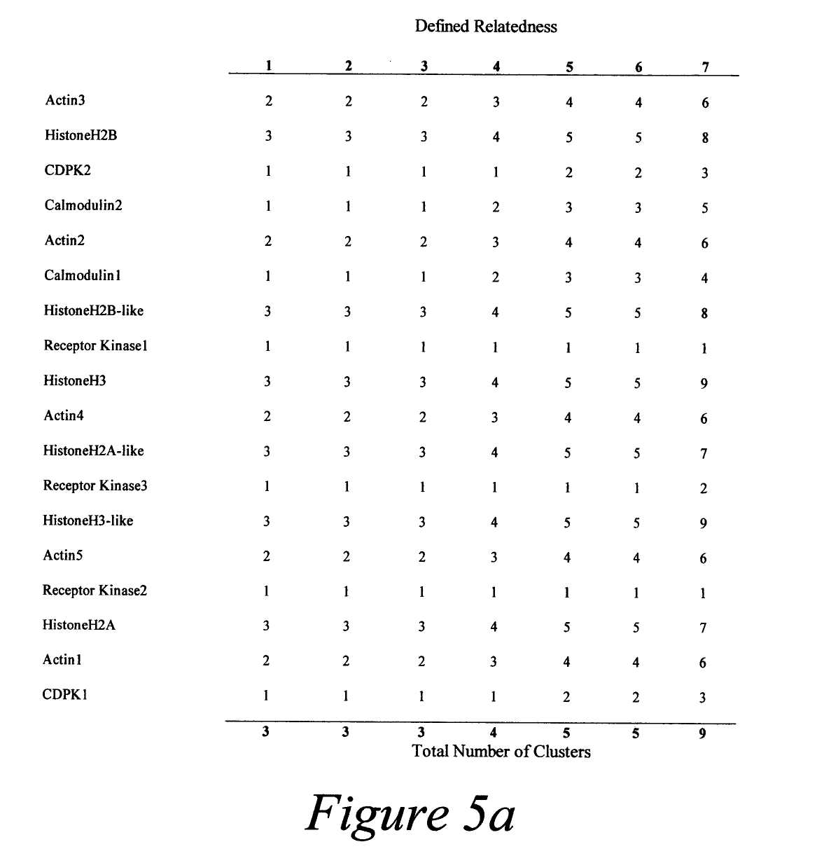

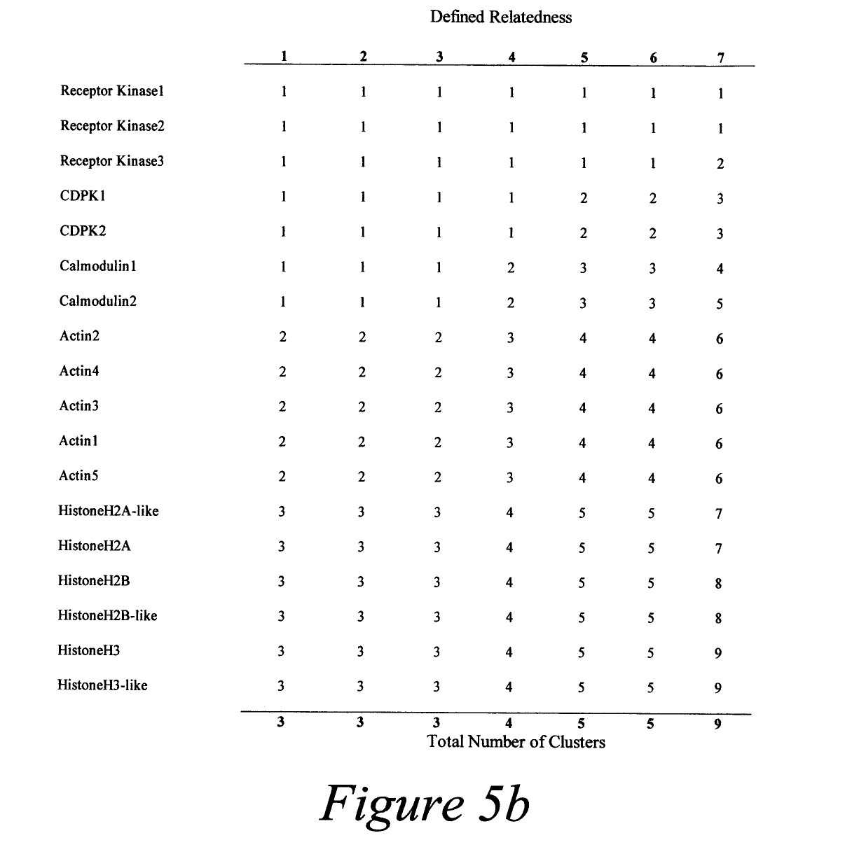

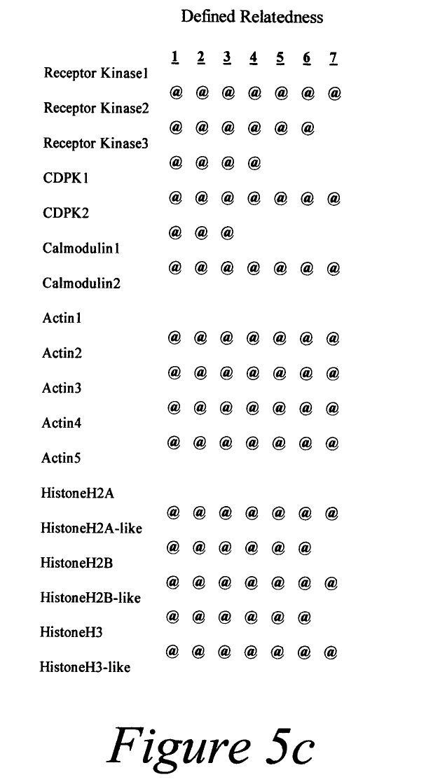

FIG. 5a shows an exemplary table of biological elements for which a comparison and clustering have already been performed. As shown in the first column of FIG. 5a, there are eighteen biological elements in the set of biological elements, and the elements are arranged in an arbitrary order. The next column, which is designated as defined relatedness “1”, identifies the cluster into which each biological element was clustered when the clustering algorithm was performed at the first level of defined relatedness. In this case, the remaining columns, marked 2-7, represent the execution of the clustering algorithm on the results of the comparison at six progressively higher levels of defined relatedness. As is evident from the table, the initial clustering at the first defined relatedness led to the generation of three clusters. The next two levels of defined relatedness (2 and 3) resulted in no new clusters being formed. At the fourth level of defined relatedness, however, a new cluster comprising CDPK2, Receptor Kinase1, Receptor Kinase3, Receptor Kinase2, and CDPK1 is formed and designated as cluster 1. Calmodulin1 and Calmodulin2, which had been part of the original cluster 1, are redesignated as forming new cluster 2. The other two clusters, which were originally clusters 2 and 3, are redesignated clusters 3 and 4, respectively. The process is repeated for defined levels of relatedness 5 through 7. At this stage of the method the biological elements have been clustered at 7 ascending levels of relatedness, and a numerical cluster designation has been given to each biological element at each of the seven levels of defined relatedness. Although numbers are given to clusters at each level of defined relatedness, for the purposes of sorting it is not required that the numbers at a given level of defined relatedness are determined or dependent upon the numbers used at any other level of defined relatedness. Rather, any cluster identification system can be used as long as the system can represent when, at any given level of defined relatedness, biological elements are in the same cluster. It is understood that alternative cluster numbering strategies can be employed that would allow the equivalent sorting.

As shown in 5b, the biological elements can now be sorted according to their clustering designations. In a preferred embodiment, the biological elements are sorted hierarchically. As shown in FIG. 5b, this can entail ordering the biological elements with priority of order given to the occurrence of lowered numbered clusters in lower numbered levels of defined relatedness. As described above, other embodiments will work equally well, depending upon the system for cluster identification used. In any case, hierarchical sorting involves the ordering of biological elements based upon clusters, with ordering occurring based on clusters from lower levels of defined relatedness to clusters at higher levels of defined relatedness. Thus, for example, receptor kinase1, which has cluster designations of “1” across all levels of defined relatedness, is sorted to the first row, followed by similarly designated receptor kinase2. This pattern is continued until all biological elements that were clustered in cluster 1 at the first defined relatedness are sorted, and then the process is repeated for the remaining original two clusters. This sorting allows the rapid hierarchical organization of biological elements according to their relatedness across a range of levels of defined relatedness. In an alternative embodiment, sorting can be performed after each application of the clustering algorithm at a new level of defined relatedness. As described herein, the method produces clusters, and increasingly refined clusters, with each more refined cluster indicating a greater level of relatedness among the biological elements in that cluster. The method therefore allows for the facile examination of a range of levels of relatedness among a variety of differentially related biological elements.

Once the sequences have been sorted, a clustergram can be generated that graphically represents the relationship between adjacent sorted biological elements. As shown in FIG. 5c, by inserting a mark, such as the “@” symbol between the cluster number results for adjacent biological elements when the two elements share a common cluster number at a given level of relatedness, a graphical representation of the relationship between adjacent biological elements can be generated. The clustergram shown in FIG. 5c visually relates the extent to which adjacent biological elements remained in the same cluster as the level of defined relatedness increased. The clustergram, or either of the tables shown in FIGS. 5a and 5b can be displayed graphically on, for example, a computer monitor.

### Implementation

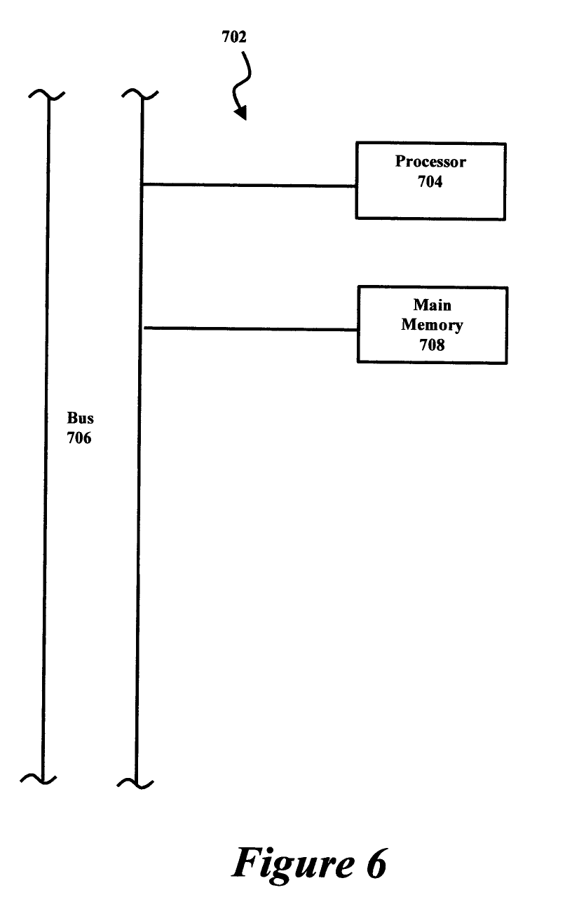

A computer system capable of carrying out the functionality and methods described above is shown in more detail in FIG. 6. A computer system 702 includes one or more processors, such as a processor 704. The processor 704 is connected to a communication bus 706. The computer system 702 also includes a main memory 708, which is preferably random access memory (RAM). Various software embodiments are described in terms of this exemplary computer system. After reading this description, it will become apparent to a person skilled in the relevant art how to implement the invention using other computer systems and/or computer architectures.

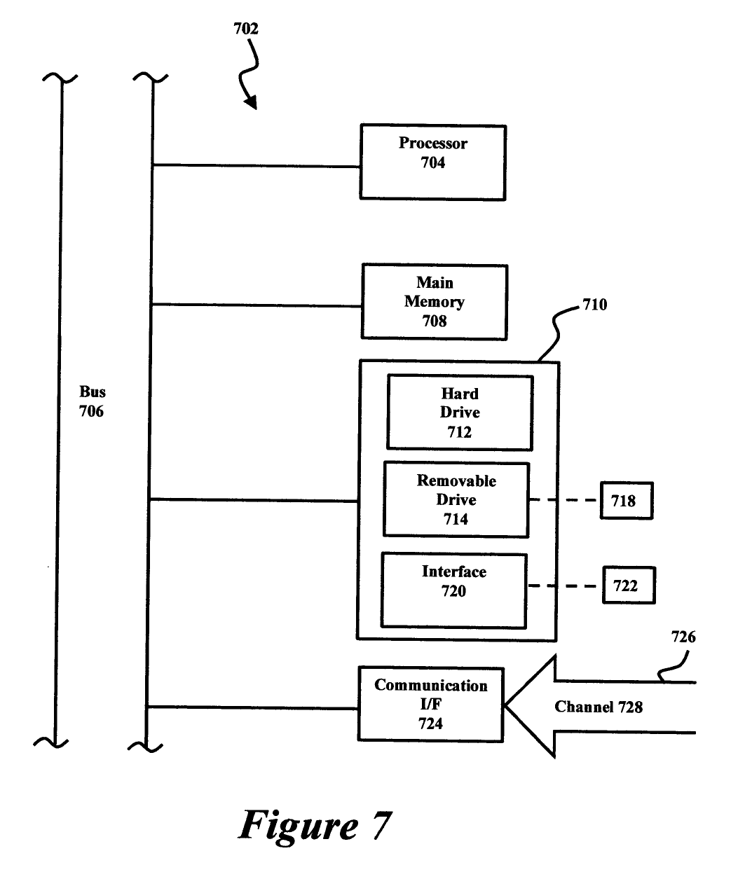

In a further embodiment, shown in FIG. 7, the computer system can also include a secondary memory 710. The secondary memory 710 can include, for example, a hard disk drive 712 and/or a removable storage drive 714, representing a floppy disk drive, a magnetic tape drive, or an optical disk drive, among others. The removable storage drive 714 reads from and/or writes to a removable storage unit 718 in a well known manner. The removable storage unit 718, represents, for example, a floppy disk, magnetic tape, or an optical disk, which is read by and written to by the removable storage drive 714. As will be appreciated, the removable storage unit 718 includes a computer usable storage medium having stored therein computer software and/or data.

In alternative embodiments, the secondary memory 710 may include other similar means for allowing computer programs or other instructions to be loaded into the computer system. Such means can include, for example, a removable storage unit 722 and an interface 720. Examples of such can include a program cartridge and cartridge interface (such as that found in video game devices), a removable memory chip (such as an EPROM, or PROM) and associated socket, and other removable storage units 722 and interfaces 720 which allow software and data to be transferred from the removable storage unit 722 to the computer system.

The computer system can also include a communications interface 724. The communications interface 724 allows software and data to be transferred between the computer system and external devices. Examples of the communications interface 724 can include a modem, a network interface (such as an Ethernet card), a communications port, a PCMCIA slot and card, etc. Software and data transferred via the communications interface 724 are in the form of signals 726 that can be electronic, electromagnetic, optical or other signals capable of being received by the communications interface 724. Signals 726 are provided to communications interface via a channel 728. A channel 728 carries signals 726 in two directions and can be implemented using wire or cable, fiber optics, a phone line, a cellular phone link, an RF link and other communications channels. In one embodiment, the channel is a connection to a network. The network can be any network known in the art, including, but not limited to, LANs, WANs, and the Internet. Nucleic acid sequence data can be stored in remote systems, databases, or distributed databases, among others, for example GenBank, and transferred to computer system for processing via the network. In a preferred embodiment, nucleic acid sequence data is received through the Internet via the channel 728. Nucleic acid sequences can be input into the system and stored in the main memory 708. Input devices include the communication and storage devices described herein, as well as keyboards, voice input, and other devices for transferring data to a computer system. In a further embodiment, nucleic acid sequences can be generated by an automatic sequencer, for example any that are known in the art, and the implementations described herein can be incorporated within the automatic sequencer device in order to directly use the output of the automatic sequencer.

In this document, the terms “computer program medium” and “computer usable medium” are used to generally refer to media such as the removable storage device 718, a hard disk installed in hard disk drive 712, and signals 726. These computer program products are means for providing software to the computer system.

Computer programs (also called computer control logic) are stored in the main memory 708 and/or the secondary memory 710. Computer programs can also be received via the communications interface 724. Such computer programs, when executed, enable the computer system to perform the features of the present invention as discussed herein. In particular, the computer programs, when executed, enable the processor 704 to perform the features of the present invention. Accordingly, such computer programs represent controllers of the computer system.

In an embodiment where the invention is implemented using software, the software may be stored in a computer program product and loaded into the computer system using the removable storage drive 714, the hard drive 712 or the communications interface 724. The control logic (software), when executed by the processor 704, causes the processor 704 to perform the functions of the invention as described herein.

In another embodiment, the invention is implemented primarily in hardware using, for example, hardware components such as application specific integrated circuits (ASICs). In one embodiment incorporating ASIC technology, a self-contained device, which could be hand-held, has integrated circuits specific to perform the methods described above without the need for software. Implementation of such a hardware state machine so as to perform the functions described herein will be apparent to persons skilled in the relevant art(s). In yet another embodiment, the invention is implemented using a combination of both hardware and software.

Each and every periodical, text, or other reference cited to herein is hereby incorporated by reference in its entirety.

The following examples are illustrative only. It is not intended that the present invention be limited to the illustrative embodiments.

### EXAMPLE 1

In this example a clustering algorithm that is capable of clustering biological elements at 100 defined relatedness levels over a range of 0.01 to 1.0 (representing 1% to 100% of query and hit in the alignment) at increments of 0.01 units is shown. This script uses data generated from “parse-blast.pl” as input. “parse-blast.pl” is a public domain software that is used to parse output of blast programs. The below example could be rewritten to accommodate input data in different formats. The script yc_cluster_inc100.pl, which is written in Perl, is shown below:

```
#!/usr/local/bin/perl −w
if ($#ARGV < 0) { die “Usage: yc_cluster_increment.pl
parse_blast.file\n”;}
$tabone = $ARGV[0];
$qry_id = $hit_id = $FR_ALQ = $FR_ALS = $cutoff = $score = “”;
for ($cutoff=0.01; $cutoff<1.01; $cutoff+=0.01){
  $cluster_no = 0;
  %cluster_ids = ( );
  %members = ( );
  @members = ( );
  open(TABO, “<$tabone”) || die “ERR \n”;
  while (<TABO>) {
    chomp;
    if ((m/\hat{ }QUERY/) || (m/\hat{ }---/)) {next;}
    @det = split (/\s+/, $_);
    $qry_id = $det[0];
    $hit_id = $det[2];
    $FR_ALQ = $det[10];
    $FR_ALS = $det[11];
    $score = $det [5];
    next if (($FR_ALQ < $cutoff) || ($FR_ALS < $cutoff) ||
    ($score < 100));
    if (defined($cluster_ids{$qry_id}) && !defined($cluster_ids
    {$hit_id})) {
      $cluster_id = $cluster_ids{$qry_id};
      $cluster_ids{$hit_id} = $cluster_id;
      push @{$members[$cluster_id]}, $hit_id;
    } elsif (defined($cluster_ids{$hit_id}) &&
!defined($cluster_ids{$qry_id})) {
      $cluster_id = $cluster_ids{$hit_id};
      $cluster_ids{$qry_id} = $cluster_id;
      push @{$members[$cluster_id]}, $qry_id;
    } elsif (defined($cluster_ids{$qry_id}) && defined
    ($cluster_ids{$hit_id}))
{
      if ($cluster_ids{$qry_id} != $cluster_ids{$hit_id}) {
        $cluster_id = $cluster_ids{$qry_id};
        $hit_cluster_id = $cluster_ids{$hit_id};
        push @{$members[$cluster_id]}, @{$members
        [$hit_cluster_id]};
        foreach( @{$members[$hit_cluster_id]} ) {
          $cluster_ids{$_} = $cluster_id;
        }
      }
    } else {
      $cluster_no++;
      $cluster_id = $cluster_no;
      $cluster_ids{$qry_id} = $cluster_id;
      $cluster_ids{$hit_id} = $cluster_id;
      push @{$members[$cluster_id]}, ($qry_id, $hit_id);
    }
  }
  close(TABO);
  while (($ID, $cluster) = each(%cluster_ids)) {
    if (defined($output{$ID})) {
      $output{$ID} .= “\t” . $cluster;
    } else {
      $output{$ID} = $cluster;
    }
  }
}
foreach $ID (keys %output){
      print “$ID\t$output{$ID}\n”;
}
```

### EXAMPLE 2

In this example a script is shown that is capable of sorting the results produced by the script of example 1 such that identical clusters are grouped together. The Perl script sort_table99.pl is shown below:

#!/usr/local/bin/perl
if ($#ARGV < 0) { die “Usage: sort_table.pl <file_name.table> #must
be tab-delimited. The table is sorted
hierarchically starting with the second, then third, then fourth column
(which contain numeric values), etc; then the entire
sorted table is printed to standard output.\n”;}
$table_name = $ARGV[0];

| | |
|---|---|
| print map { $_->[0] } | # after sorting prints whole |
| line | |
| sort { | |
| $a->[1] <=> $b->[1] | # sorts second column |
| \|\| | |
| $a->[2] <=> $b->[2] | # sorts third column |
| \|\| | |
| $a->[3] <=> $b->[3] | # sorts fourth column |
| \|\| | |
| $a->[4] <=> $b->[4] | # etc |
| \|\| | |
| $a->[5] <=> $b->[5] | |
| \|\| | |
| $a->[6] <=> $b->[6] | |
| \|\| | |
| $a->[7] <=> $b->[7] | |
| \|\| | |
| $a->[8] <=> $b->[8] | |
| \|\| | |
| $a->[9] <=> $b->[9] | |
| \|\| | |
| $a->[10] <=> $b->[10] | |
| \|\| | |
| $a->[11] <=> $b->[11] | |
| \|\| | |
| $a->[12] <=> $b->[12] | |
| \|\| | |
| $a->[13] <=> $b->[13] | |
| \|\| | |
| $a->[14] <=> $b->[14] | |
| \|\| | |
| $a->[15] <=> $b->[15] | |
| \|\| | |
| $a->[16] <=> $b->[16] | |
| \|\| | |
| $a->[17] <=> $b->[17] | |
| \|\| | |
| $a->[18] <=> $b->[18] | |
| \|\| | |
| $a->[19] <=> $b->[19] | |
| \|\| | |
| $a->[20] <=> $b->[20] | |
| \|\| | |
| $a->[21] <=> $b->[21] | |
| \|\| | |
| $a->[22] <=> $b->[22] | |
| \|\| | |
| $a->[23] <=> $b->[23] | |
| \|\| | |
| $a->[24] <=> $b->[24] | |
| \|\| | |
| $a->[25] <=> $b->[25] | |
| \|\| | |
| $a->[26] <=> $b->[26] | |
| \|\| | |
| $a->[27] <=> $b->[27] | |
| \|\| | |
| $a->[28] <=> $b->[28] | |
| \|\| | |
| $a->[29] <=> $b->[29] | |
| \|\| | |
| $a->[30] <=> $b->[30] | |
| \|\| | |
| $a->[31] <=> $b->[31] | |
| \|\| | |
| $a->[32] <=> $b->[32] | |
| \|\| | |
| $a->[33] <=> $b->[33] | |
| \|\| | |
| $a->[34] <=> $b->[34] | |
| \|\| | |
| $a->[35] <=> $b->[35] | |
| \|\| | |
| $a->[36] <=> $b->[36] | |
| \|\| | |
| $a->[37] <=> $b->[37] | |
| \|\| | |
| $a->[38] <=> $b->[38] | |
| \|\| | |
| $a->[39] <=> $b->[39] | |
| \|\| | |
| $a->[40] <=> $b->[40] | |
| \|\| | |
| $a->[41] <=> $b->[41] | |
| \|\| | |
| $a->[42] <=> $b->[42] | |
| \|\| | |
| $a->[43] <=> $b->[43] | |
| \|\| | |
| $a->[44] <=> $b->[44] | |
| \|\| | |
| $a->[45] <=> $b->[45] | |
| \|\| | |
| $a->[46] <=> $b->[46] | |
| \|\| | |
| $a->[47] <=> $b->[47] | |
| \|\| | |
| $a->[48] <=> $b->[48] | |
| \|\| | |
| $a->[49] <=> $b->[49] | |
| \|\| | |
| $a->[50] <=> $b->[50] | |
| \|\| | |
| $a->[51] <=> $b->[51] | |
| \|\| | |
| $a->[52] <=> $b->[52] | |
| \|\| | |
| $a->[53] <=> $b->[53] | |
| \|\| | |
| $a->[54] <=> $b->[54] | |
| \|\| | |
| $a->[55] <=> $b->[55] | |
| \|\| | |
| $a->[56] <=> $b->[56] | |
| \|\| | |
| $a->[57] <=> $b->[57] | |
| \|\| | |
| $a->[58] <=> $b->[58] | |
| \|\| | |
| $a->[59] <=> $b->[59] | |
| \|\| | |
| $a->[60] <=> $b->[60] | |
| \|\| | |
| $a->[61] <=> $b->[61] | |
| \|\| | |
| $a->[62] <=> $b->[62] | |
| \|\| | |
| $a->[63] <=> $b->[63] | |
| \|\| | |
| $a->[64] <=> $b->[64] | |
| \|\| | |
| $a->[65] <=> $b->[65] | |
| \|\| | |
| $a->[66] <=> $b->[66] | |
| \|\| | |
| $a->[67] <=> $b->[67] | |
| \|\| | |
| $a->[68] <=> $b->[68] | |
| \|\| | |
| $a->[69] <=> $b->[69] | |
| \|\| | |
| $a->[70] <=> $b->[70] | |
| \|\| | |
| $a->[71] <=> $b->[71] | |
| \|\| | |
| $a->[72] <=> $b->[72] | |
| \|\| | |
| $a->[73] <=> $b->[73] | |
| \|\| | |
| $a->[74] <=> $b->[74] | |
| \|\| | |
| $a->[75] <=> $b->[75] | |
| \|\| | |
| $a->[76] <=> $b->[76] | |
| \|\| | |
| $a->[77] <=> $b->[77] | |
| \|\| | |
| $a->[78] <=> $b->[78] | |
| \|\| | |
| $a->[79] <=> $b->[79] | |
| \|\| | |
| $a->[80] <=> $b->[80] | |
| \|\| | |
| $a->[81] <=> $b->[81] | |
| \|\| | |
| $a->[82] <=> $b->[82] | |
| \|\| | |
| $a->[83] <=> $b->[83] | |
| \|\| | |
| $a->[84] <=> $b->[84] | |
| \|\| | |
| $a->[85] <=> $b->[85] | |
| \|\| | |
| $a->[86] <=> $b->[86] | |
| \|\| | |
| $a->[87] <=> $b->[87] | |
| \|\| | |
| $a->[88] <=> $b->[88] | |
| \|\| | |
| $a->[89] <=> $b->[89] | |
| \|\| | |
| $a->[90] <=> $b->[90] | |
| \|\| | |
| $a->[91] <=> $b->[91] | |
| \|\| | |
| $a->[92] <=> $b->[92] | |
| \|\| | |
| $a->[93] <=> $b->[93] | |
| \|\| | |
| $a->[94] <=> $b->[94] | |
| \|\| | |
| $a->[95] <=> $b->[95] | |
| \|\| | |
| $a->[96] <=> $b->[96] | |
| \|\| | |
| $a->[97] <=> $b->[97] | |
| \|\| | |
| $a->[98] <=> $b->[98] | |
| \|\| | |
| $a->[99] <=> $b->[99] | |
| } | |
| map { [ $_, (split /\s+/) | |

[1,2,3,4,5,6,7,8,9,10,11,12,13,14,15,16,17,18,19,20,21,22,23,24,25,26,
27,28,29,30,31,32,33,34,35,36,37,38,39,40,41,42,43,44,45,46,47,48,49,
50,51,52,53,54,55,56,57,58,59,60,61,62,63,64,65,66,67,68,69,70,71,72,
73,74,75,76,77,78,79,80,81,82,83,84,85,86,87,88,89,90,91,92,93,94,95,
96,97,98,99] ] } # puts columns into a mapped array
‘cat $table_name‘; # calls system to read
specified file_name.table

### EXAMPLE 3

In this example a script that is capable of graphically displaying the results of the script of example 2 is shown. In this script an “n” is used to symbolize membership of adjacent sequences in a common cluster number. When the output is imported into an Excel spreadsheet, the “n” is displayed as a dot symbol in the Marlett font. The Perl script clustergram99.pl is shown below:

```
#!/usr/local/bin/perl −w
if ($#ARGV < 0) { die “file1 file2
$tableone = $ARGV[0];
#%hash = ( );
open(TAB, “<$tableone”) || die “Cannot open $tableone \n”;
while (<TAB>) {
  $line = $_;
  chomp $line;
  ($ID, @array) = split (/\t/, $line);
    push (@IDs, $ID);
  $hash1{$ID} = $array[0];
  $hash2{$ID} = $array[1];
  $hash3{$ID} = $array[2];
  $hash4{$ID} = $array[3];
  $hash5{$ID} = $array[4];
  $hash6{$ID} = $array[5];
  $hash7{$ID} = $array[6];
  $hash8{$ID} = $array[7];
  $hash9{$ID} = $array[8];
  $hash10{$ID} = $array[9];
  $hash11{$ID} = $array[10];
  $hash12{$ID} = $array[11];
  $hash13{$ID} = $array[12];
  $hash14{$ID} = $array[13];
  $hash15{$ID} = $array[14];
  $hash16{$ID} = $array[15];
  $hash17{$ID} = $array[16];
  $hash18{$ID} = $array[17];
  $hash19{$ID} = $array[18];
  $hash20{$ID} = $array[19];
  $hash21{$ID} = $array[20];
  $hash22{$ID} = $array[21];
  $hash23{$ID} = $array[22];
  $hash24{$ID} = $array[23];
  $hash25{$ID} = $array[24];
  $hash26{$ID} = $array[25];
  $hash27{$ID} = $array[26];
  $hash28{$ID} = $array[27];
  $hash29{$ID} = $array[28];
  $hash30{$ID} = $array[29];
  $hash31{$ID} = $array[30];
  $hash32{$ID} = $array[31];
  $hash33{$ID} = $array[32];
  $hash34{$ID} = $array[33];
  $hash35{$ID} = $array[34];
  $hash36{$ID} = $array[35];
  $hash37{$ID} = $array[36];
  $hash38{$ID} = $array[37];
  $hash39{$ID} = $array[38];
  $hash40{$ID} = $array[39];
  $hash41{$ID} = $array[40];
  $hash42{$ID} = $array[41];
  $hash43{$ID} = $array[42];
  $hash44{$ID} = $array[43];
  $hash45{$ID} = $array[44];
  $hash46{$ID} = $array[45];
  $hash47{$ID} = $array[46];
  $hash48{$ID} = $array[47];
  $hash49{$ID} = $array[48];
  $hash50{$ID} = $array[49];
  $hash51{$ID} = $array[50];
  $hash52{$ID} = $array[51];
  $hash53{$ID} = $array[52];
  $hash54{$ID} = $array[53];
  $hash55{$ID} = $array[54];
  $hash56{$ID} = $array[55];
  $hash57{$ID} = $array[56];
  $hash58{$ID} = $array[57];
  $hash59{$ID} = $array[58];
  $hash60{$ID} = $array[59];
  $hash61{$ID} = $array[60];
  $hash62{$ID} = $array[61];
  $hash63{$ID} = $array[62];
  $hash64{$ID} = $array[63];
  $hash65{$ID} = $array[64];
  $hash66{$ID} = $array[65];
  $hash67{$ID} = $array[66];
  $hash68{$ID} = $array[67];
  $hash69{$ID} = $array[68];
  $hash70{$ID} = $array[69];
  $hash71{$ID} = $array[70];
  $hash72{$ID} = $array[71];
  $hash73{$ID} = $array[72];
  $hash74{$ID} = $array[73];
  $hash75{$ID} = $array[74];
  $hash76{$ID} = $array[75];
  $hash77{$ID} = $array[76];
  $hash78{$ID} = $array[77];
  $hash79{$ID} = $array[78];
  $hash80{$ID} = $array[79];
  $hash81{$ID} = $array[80];
  $hash82{$ID} = $array[81];
  $hash83{$ID} = $array[82];
  $hash84{$ID} = $array[83];
  $hash85{$ID} = $array[84];
  $hash86{$ID} = $array[85];
  $hash87{$ID} = $array[86];
  $hash88{$ID} = $array[87];
  $hash89{$ID} = $array[88];
  $hash90{$ID} = $array[89];
  $hash91{$ID} = $array[90];
  $hash92{$ID} = $array[91];
  $hash93{$ID} = $array[92];
  $hash94{$ID} = $array[93];
  $hash95{$ID} = $array[94];
  $hash96{$ID} = $array[95];
  $hash97{$ID} = $array[96];
  $hash98{$ID} = $array[97];
  $hash99{$ID} = $array[98];
  }
  close(TAB);
  for ($i=0; $i<@IDs; $i++) {
    $n = $i + 1;
    $ID1 = $IDs[$i];
    $ID2 = $IDs[$n];
    print “$ID1\n”;
    print “\t”;
    if ($hash1{$ID1} == $hash1{$ID2}){
      print “n\t”;
      if ($hash2{$ID1} == $hash2{$ID2}){
        print “n\t”;
      if ($hash3{$ID1} == $hash3{$ID2}){
        print “n\t”;
        if ($hash4{$ID1} == $hash4{$ID2}){
          print “n\t”;
          if ($hash5{$ID1} == $hash5{$ID2}){
            print “n\t”;
            if ($hash6{$ID1} == $hash6{$ID2}){
              print “n\t”;
            if ($hash7{$ID1} == $hash7{$ID2}){
              print “n\t”;
              if ($hash8{$ID1} == $hash8{$ID2}){
                print “n\t”;
                if ($hash9{$ID1} == $hash9{$ID2}){
                  print “n\t”;
                  if ($hash10{$ID1} == $hash10{$ID2}){
                  print “n\t”;
                  if ($hash11{$ID1} == $hash11{$ID2}){
                    print “n\t”;
                    if ($hash12{$ID1} == $hash12{$ID2}){
                    print “n\t”;
                    if ($hash13{$ID1} == $hash13{$ID2}){
                      print “n\t”;
                      if ($hash14{$ID1} == $hash14{$ID2}){
                      print “n\t”;
                      if ($hash15{$ID1} == $hash15{$ID2}){
                        print “n\t”;
                        if ($hash16{$ID1} == $hash16{$ID2}){
                          print “n\t”;
                          if ($hash17{$ID1} == $hash17{$ID2}){
                            print “n\t”;
                            if ($hash18{$ID1} == $hash18{$ID2}){
                            print “n\t”;
                            if ($hash19{$ID1} == $hash19{$ID2}){
                              print “n\t”;
                              if ($hash20{$ID1} == $hash20{$ID2}){
                                print “n\t”;
                                if ($hash21{$ID1} == $hash21{$ID2}){
                                  print “n\t”;
                                  if ($hash22{$ID1} == $hash22{$ID2}){
                                  print “n\t”;
                                  if ($hash23{$ID1} == $hash23{$ID2}){
    print “n\t”;
    if ($hash24{$ID1} == $hash24{$ID2}){
      print “n\t”;
      if ($hash25{$ID1} == $hash25{$ID2}){
      print “n\t”;
      if ($hash26{$ID1} == $hash26{$ID2}){
        print “n\t”;
        if ($hash27{$ID1} == $hash27{$ID2}){
          print “n\t”;
          if ($hash28{$ID1} == $hash28{$ID2}){
            print “n\t”;
            if ($hash29{$ID1} == $hash29{$ID2}){
            print “n\t”;
            if ($hash30{$ID1} == $hash30{$ID2}){
              print “n\t”;
              if ($hash31{$ID1} == $hash31{$ID2}){
                print “n\t”;
                if ($hash32{$ID1} == $hash32{$ID2}){
                  print “n\t”;
                  if ($hash33{$ID1} == $hash33{$ID2}){
                  print “n\t”;
                  if ($hash34{$ID1} == $hash34{$ID2}){
                    print “n\t”;
                    if ($hash35{$ID1} == $hash35{$ID2}){
                      print “n\t”;
                      if ($hash36{$ID1} == $hash36{$ID2}){
                        print “n\t”;
                        if ($hash37{$ID1} == $hash37{$ID2}){
                        print “n\t”;
                        if ($hash38{$ID1} == $hash38{$ID2}){
                          print “n\t”;
                          if ($hash39{$ID1} == $hash39{$ID2}){
                            print “n\t”;
                            if ($hash40{$ID1} == $hash40{$ID2}){
                              print “n\t”;
                              if ($hash41{$ID1} == $hash41{$ID2}){
                              print “n\t”;
                              if ($hash42{$ID1} == $hash42{$ID2}){
                                print “n\t”;
                                if ($hash43{$ID1} == $hash43{$ID2}){
                                  print “n\t”;
                                  if ($hash44{$ID1} == $hash44{$ID2}){
                                    print “n\t”;
    if ($hash45{$ID1} == $hash45{$ID2}){
      print “n\t”;
      if ($hash46{$ID1} == $hash46{$ID2}){
        print “n\t”;
      if ($hash47{$ID1} == $hash47{$ID2}){
        print “n\t”;
        if ($hash48{$ID1} == $hash48{$ID2}){
          print “n\t”;
          if ($hash49{$ID1} == $hash49{$ID2}){
            print “n\t”;
            if ($hash50{$ID1} == $hash50{$ID2}){
              print “n\t”;
            if ($hash51{$ID1} == $hash51{$ID2}){
              print “n\t”;
              if ($hash52{$ID1} == $hash52{$ID2}){
                print “n\t”;
                if ($hash53{$ID1} == $hash53{$ID2}){
                  print “n\t”;
                  if ($hash54{$ID1} == $hash54{$ID2}){
                  print “n\t”;
                  if ($hash55{$ID1} == $hash55{$ID2}){
                    print “n\t”;
                    if ($hash56{$ID1} == $hash56{$ID2}){
                    print “n\t”;
                    if ($hash57{$ID1} == $hash57{$ID2}){
                      print “n\t”;
                      if ($hash58{$ID1} == $hash58{$ID2}){
                      print “n\t”;
                      if ($hash59{$ID1} == $hash59{$ID2}){
                        print “n\t”;
                        if ($hash60{$ID1} == $hash60{$ID2}){
                          print “n\t”;
                          if ($hash61{$ID1} == $hash61{$ID2}){
                            print “n\t”;
                            if ($hash62{$ID1} == $hash62{$ID2}){
                            print “n\t”;
                            if ($hash63{$ID1} == $hash63{$ID2}){
                              print “n\t”;
                              if ($hash64{$ID1} == $hash64{$ID2}){
                                print “n\t”;
                                if ($hash65{$ID1} == $hash65{$ID2}){
                                  print “n\t”;
                                  if ($hash66{$ID1} == $hash66{$ID2}){
                                  print “n\t”;
                                  if ($hash67{$ID1} == $hash67{$ID2}){
    print “n\t”;
    if ($hash68{$ID1} == $hash68{$ID2}){
      print “n\t”;
      if ($hash69{$ID1} == $hash69{$ID2}){
      print “n\t”;
      if ($hash70{$ID1} == $hash70{$ID2}){
        print “n\t”;
        if ($hash71{$ID1} == $hash71{$ID2}){
          print “n\t”;
          if ($hash72{$ID1} == $hash72{$ID2}){
            print “n\t”;
            if ($hash73{$ID1} == $hash73{$ID2}){
            print “n\t”;
            if ($hash74{$ID1} == $hash74{$ID2}){
              print “n\t”;
              if ($hash75{$ID1} == $hash75{$ID2}){
                print “n\t”;
                if ($hash76{$ID1} == $hash76{$ID2}){
                  print “n\t”;
                  if ($hash77{$ID1} == $hash77{$ID2}){
                  print “n\t”;
                  if ($hash78{$ID1} == $hash78{$ID2}){
                    print “n\t”;
                    if ($hash79{$ID1} == $hash79{$ID2}){
                      print “n\t”;
                      if ($hash80{$ID1} == $hash80{$ID2}){
                        print “n\t”;
                        if ($hash81{$ID1} == $hash81{$ID2}){
                        print “n\t”;
                        if ($hash82{$ID1} == $hash82{$ID2}){
                          print “n\t”;
                          if ($hash83{$ID1} == $hash83{$ID2}){
                            print “n\t”;
                            if ($hash84{$ID1} == $hash84{$ID2}){
                              print “n\t”;
                              if ($hash85{$ID1} == $hash85{$ID2}){
                              print “n\t”;
                              if ($hash86{$ID1} == $hash86{$ID2}){
                                print “n\t”;
                                if ($hash87{$ID1} == $hash87{$ID2}){
                                  print “n\t”;
                                  if ($hash88{$ID1} == $hash88{$ID2}){
                                    print “n\t”;
                                    if ($hash89{$ID1} == $hash89{$ID2}){
                                      print “n\t”;
                                      if ($hash90{$ID1} == $hash90{$ID2}){
                                        print “n\t”;
                                        if ($hash91{$ID1} == $hash91{$ID2}){
                                        print “n\t”;
                                        if ($hash92{$ID1} == $hash92{$ID2}){
                                          print “n\t”;
                                          if ($hash93{$ID1} == $hash93{$ID2}){
                                            print “n\t”;
                                            if ($hash94{$ID1} == $hash94{$ID2}){
                                              print “n\t”;
                                              if ($hash95{$ID1} == $hash95{$ID2}){
                                              print “n\t”;
                                              if ($hash96{$ID1} == $hash96{$ID2}){
                                                print “n\t”;
                                                if ($hash97{$ID1} == $hash97{$ID2}){
                                                  print “n\t”;
                                                  if ($hash98{$ID1} == $hash98{$ID2}){
                                                    print “n\t”;
                                                      if ($hash99{$ID1} == $hash89{$ID2}){
                                                        print “n\t”;
}}}}}}}}}}}}}}}}}}}}}}}}}}}}}}}}}}}}}}}}}}}}}}}}}}}}}}}}}}}}}}}}}}}}}}}}}}}}}}
}}}}}}}}}}}}}}}}}}}}}
    print “\n”;
    }
```

### EXAMPLE 4

This example demonstrates utilizes the scripts of examples 1, 2, and 3 to organize protein sequences into clusters. An Arabidopsis protein sequence database is searched for sequences containing the following keywords in their description lines: “P450”, “sugar transporter”, “calmodulin”, “ABC”, and “phosphatase 2C”. Eight sequences from each keyword search are chosen for analysis, resulting in a total of 40 sequences in the test set (SEQ ID Nos: 1-40). Each sequence in the test set is used as a query to search the entire test set using blastp (version 2.0.14) (an all-versus-all analysis).

The blastp output is parsed using the public domain software “parse_blast.pl” with the “-table” parameter set to “2”. The output of “parse_blast.pl” is used as input for the script “yc_cluster_inc100.pl”. The output of “yc_cluster_inc100.pl is used as input for the script “sort_table99.pl”. The output of “sort_table99.pl” is used as input for the script “clustergram99.pl”. The output of “clustergram99.pl” is imported into Microsoft Excel 2000 (Microsoft Corporation, version 9.0.3821 SR-1) for viewing. Data in columns B through CW is changed to font “Marlett” and centered within the cells in order to improve graphic appearance.

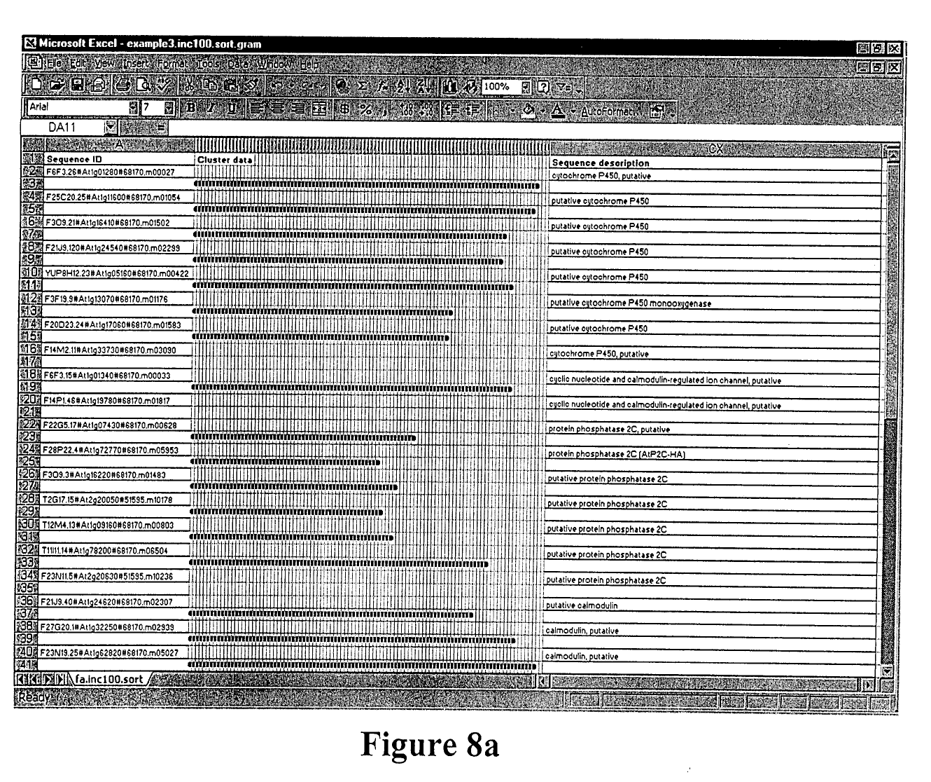

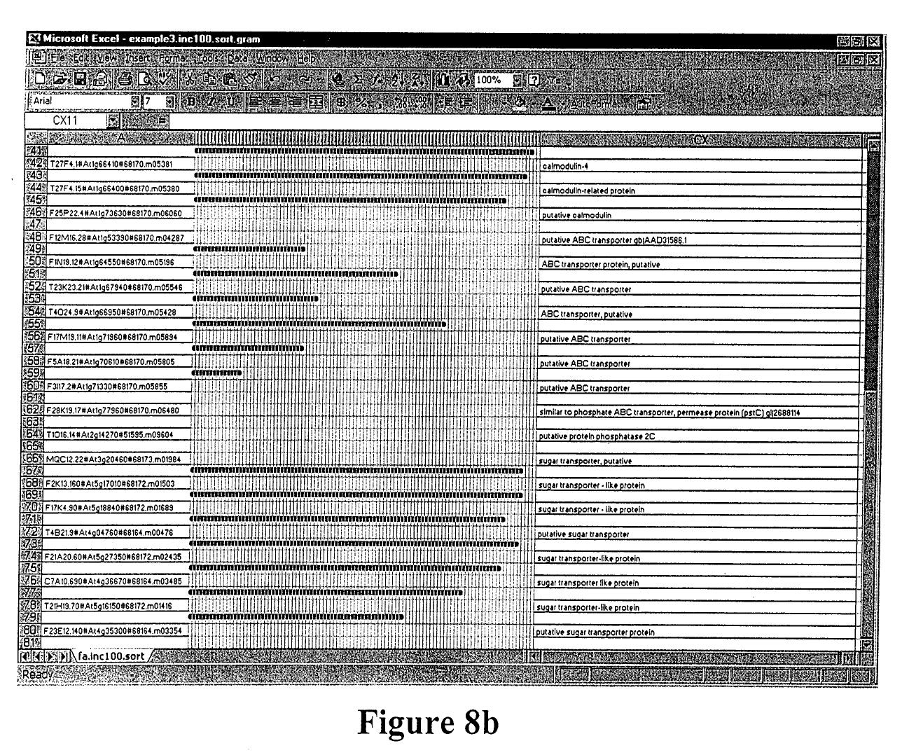

The clustergram (FIGS. 8a and 8b) graphically displays incremental clustering data. Membership of sequences listed in column A in common clusters is indicated by the presence of a dot in the odd-numbered rows in columns B through CW between even-numbered sequence rows. Cluster relatedness is increased in each column (columns B through CW) from 0.01 to 1.0 fraction of query and hit lengths in the blastp alignment. Thus, the sequences indicated on lines 14 and 16 are co-clustered through 72 levels of cluster stringency but are not co-clustered at higher levels of stringency (i.e. at or above 0.73 fraction of query and hit in the blastp alignment). Absence of a dot in any column of columns B through CW between two rows containing sequences indicates that the two sequences did not cluster even at the lowest level of stringency. Thus, no relationship was found between the two sequences indicated on lines 16 and 18, for example. Examination of the sequence descriptions (column CX) indicates a correlation between descriptions and membership within clusters. Thus, the eight sequences described as being cytochrome P450 genes, in lines 2, 4, 6, 8, 10, 12, 14, and 16, are all co-clustered, and are not clustered with genes of any of the other described families.

This example successfully distinguishes between two unrelated gene families: the cyclic nucleotide/calmodulin-regulated ion channel family (FIG. 8a), and the calmodulin family (FIGS. 8a and 8b), which were both selected for this analysis due to the presence of the keyword “calmodulin” in the sequence description lines. Further, sequences listed in lines 62 and 64 did not co-cluster with any other sequence in the test set, despite having sequence descriptions that are very similar to others in the set. Specifically, sequence SEQ ID NO: 28 described as an ABC transporter, did not co-cluster with other ABC transporters within this test set, and sequence SEQ ID NO: 30, described as a protein phosphatase 2C, did not co-cluster with other protein phosphatase 2C genes within this test set. However, examination of the raw blastp output data for these two genes reveals that neither of these sequences are significantly related to any of the other sequences within the test set. Thus, this example appropriately assigned these two genes to two distinct clusters, in which they are sole members.

1. A method of analyzing a set of DNA sequences comprising:
a) performing an all-versus-all comparison of said set;
b) applying a transitive clustering algorithm at a defined relatedness to said set using results of said comparison to produce one or more clusters;
c) repeating step b) one or more times at increasingly greater levels of relatedness;
d) sorting the DNA sequences in a hierarchy based on said clusters; and
e) displaying the sorted DNA sequences;
wherein said defined relatedness is a value derived from a member of the group consisting of percent identity percent similarity, e-value, bit score and fraction of query and hit.

2-11. (canceled)

12. A program storage device readable by a machine, tangibly embodying a program of instructions executable by a machine to perform method steps to analyze a set of DNA sequences comprising:
a) performing all-versus-all comparison of said set including parsing said sequences using software that substantially follows the steps of the public domain Perl script “parse-blast.pl”:
b) applying a transitive clustering algorithm at a defined relatedness to said set using results of said comparison to produce one or more clusters where said algorithm substantially follows the steps of the Perl script “yc_cluster_inc100.pl”;
c) repeating step b) one or more times at increasingly greater levels of relatedness;
d) sorting said DNA sequences in a hierarchy based on said clusters where said sorting substantially follows the steps of the Perl script “sort_table99.pl”, and
e) displaying the sorted DNA sequences using software that substantially follows the Perl script “clustergram99.pl”.

13-21. (canceled)
